(19) United States
(12) Patent Application Publication
(10) Pub. No.: US 2003/0134263 A1
(43) Pub. Date: Jul. 17, 2003

(54) Regulatory nucleic acid assay for diagnostic and library screens

(76) Inventor: Albert J. Erives, Kensington, CA (US)

Correspondence Address: MORRISON & FOERSTER LLP, 3811 VALLEY CENTRE DRIVE, SUITE 500, SAN DIEGO, CA, 92130-2332, US

(21) Appl. No.: 09/989,993

(22) Filed: Nov. 21, 2001

(51) Int. Cl. C12Q 1/00; C12Q 1/68; C07H 21/04; C12P 21/02; C12N 5/06; C07K 14/435

(52) U.S. Cl. 435/4; 435/6; 435/69.1; 435/320.1; 435/325; 530/350; 536/23.1

(57) ABSTRACT

The present invention relates generally to compositions and use of the compositions to screen for compounds which are able to regulate the expression of desired genes.

### TECHNICAL FIELD

The present invention relates to the construction and use of regulatory nucleic acid sequences to study the effects of compounds in various tissues and disease conditions.

### INTRODUCTION

The invention provides a method to screen for lead compounds which are able to regulate expression of genes whose expression levels are correlated with either beneficial or deleterious effects on cell metabolism or growth. The compounds may directly affect transcription by interaction with expression control elements, or may affect signaling pathways which ultimately interact with expression control elements in the gene. Of particular relevance to the present invention are the control elements known as enhancers.

Many signaling pathways exist in cells. For example, one such pathway may begin with a ligand interacting with a cell surface transmembrane spanning receptor, initiating an intracellular cascade of events in which various signaling molecules, such as kinases and phosphorylases, are involved, and resulting in the activation of nuclear transcription factors which act upon sequences called enhancer regions. These enhancer regions can then regulate the activation of transcription from a promoter that controls transcription of a specific gene or set of genes. Each pathway is complicated and involves many players. In addition, each pathway can interact with other pathways.

In order to alter the transcription of a gene, it is often necessary to dissect the entire pathway in which the gene is involved or portions of it. If it is known that a specific enhancer or other sequence involved in the pathway regulates the transcription of a particular gene, then experiments can be designed, without knowing all the players, to test what conditions result in either increased, or decreased transcription of the gene. It is not necessary to know the pathways involved. It is only necessary to know that the enhancer works regulating transcription of a specific gene or genes in a specific cell type or cell physiological state. A cell is then chosen that endogenously expresses or is competent to induce a gene of choice, for example, erythropoietin (EPO), and that has the pathway that controls expression or induction of the selected gene. A vector containing the EPO enhancer operably linked to a basal promoter, that drives the expression of a reporter protein, can be placed into the cell. In the same vector, a second enhancer element, different than the first, operably linked to a promoter controls expression of a second reporter gene different than the first reporter gene. This second enhancer, promoter, and reporter gene serve as a control during a screen. An insulator is placed between the first enhancer, promoter, reporter complex, and the second enhancer, promoter, reporter complex. Compounds that only change the transcription and subsequent expression of the reporter protein downstream of the target enhancer (the EPO enhancer), are selected as positives for further screening. Whereas compounds that affect expression of both reporter genes will not be selected as they act nonspecifically. Therefore, a cell can be contacted with a test compound that can act through any of the players in the pathway that controls expression of EPO, resulting in the activation or inhibition of the enhancer, which then interacts with the basal promoter resulting in the expression of the reporter protein.

The arrangement of elements described above allow screens to be conducted for regulators or modulators of the known or unknown players in the pathway controlling expression of the gene or genes of choice using a single compound or a compound library. As described above, a test compound can be placed into a cell, and that compound could interact with and/or affect any step in the pathway thereby controlling transcription through the enhancer.

The pathology of a disease often involves the misexpression of a gene or genes. Therefore, it would be advantageous to fix the misexpression of a gene. This could lead to the reduction of the diseases's effects of or the elimination of the disease. If a small molecule could be found, whose actions result in the altered activity of an enhancer that is known to act on a promoter of a gene that is misexpressed, then the misexpression of the gene could be corrected.

For example, it would be useful to conduct a screen for a small molecule that functions to upregulate the endogenous expression of a protein. Increased expression of the protein could result in the elimination of the cause of a disease if, for example, the disease was a result of decreased levels of the protein being made by the subject. Alternatively, it also would be useful to conduct a screen for a compound that functions to inhibit expression of a protein that might be overexpressed in a disease.

A variety of compound libraries can be screened to obtain a “lead” compound to be used in the treatment of a particular disease. Then, the “lead” compound or a known drug, useful for the regulation of a selected gene, can be altered, by changing its chemical side groups for example, to make it more specific in its action. This altered compound can be exposed to cells which express the selected gene and its effects analysed.

Any gene specific enhancer linked to a reporter gene can be used in the invention. Any enhancer can be used as long as it is known what gene or genes it transcriptionally regulates. The enhancer can be an endogenous enhancer that regulates the transcription of a gene in a normal healthy cell. Or the enhancer can be an endogenous enhancer that regulates the transcription of a gene in a cell exposed to certain conditions or stresses that are not normally found in a healthy cell.

An endogenous enhancer, by interacting with a basal promoter for a specific gene, can regulate the transcription of that gene or several genes in a normal healthy cell. If it is known that an enhancer regulates the transcription of a gene, then that transcription can be increased or decreased by modulating the pathway activating the enhancer.

An example of a situation in which a cell is under abnormal stress is when there is a reduction of oxygen supply below normal physiological levels; this situation is known as a state of hypoxia. Protein expression in cells changes as the cell is exposed to different conditions. For example, low or normal levels of EPO are found in cells when physiological oxygen levels are present. But when oxygen levels drop, expression of EPO is upregulated through activation of the EPO gene specific enhancer. Erythropoietin is a glycoprotein (46 kD) hormone produced by specialized cells in the kidneys that regulate the production of red blood cells in the marrow. These cells are sensitive to low arterial oxygen concentration and will release erythropoietin when oxygen is low. Erythropoietin stimulates the bone marrow to produce more red blood cells (to increase the oxygen caring capacity of the blood).

It would be useful to screen for compounds that upregulate the expression of endogenous erythropoietin (EPO). These compounds could be used to treat anemia, the condition in which too few blood cells exist in the bloodstream, resulting in insufficient oxygen to tissues and organs.

The level of this hormone in the bloodstream can indicate bone marrow disorders or kidney disease. Normal levels of erythropoietin are 0 to 19 mU/ml (milliunits per millilitre). Elevated levels can be seen, for example, in polycythaemia rubra vera. Lower than normal values are seen in chronic renal failure. Recombinant erythopoeitin is now being used therapeutically in patients.

Under conditions of low oxygen levels, the transcription of certain genes are induced by regulatory sequences, such as enhancers, that act as hypoxia sensors. These genes sometimes encode endothelial growth factors, which cause blood vessel growth into the region of low oxygen concentrations. Though in a healthy cell, these genes are often used during development and in tissue-repair, these genes are often exploited in oncogenesis.

One thing that determines tumor growth is vascularization or the growth of blood vessels into a tissue mass, resulting in the needed oxygen and nutrient supply to support growth of the tumor. As a tumor grows, oxygen levels decrease. During low levels of oxygen, vascular endothelial growth factor (VEGF) expression increases. This increased expression of VEGF results in increased vascularization allowing the tumor to continue to grow. Therefore, if the expression of an endothelial growth factor can be controlled, or not induced under conditions of low oxygen, a lack of vascularization and a decrease in or halting of the growth of the tumor could result. It would be useful to screen for compounds that down regulate VEGF. These compounds could be used to treat cancer by inhibiting angiogenesis, the process of vascularization of a tissue involving the development of new capillary blood vessels. Angiogenesis is stimulated by VEGF acting via endothelial cell-specific receptors, such as VEGFR-2, that are overexpressed at the sites of angiogenesis.

In addition, diabetic retinopathy is characterized by an increased retinal neovascularization due to the action of VEGF. Thus, compounds could be screened for and used to treat diabetic retinopathy.

Diabetic retinopathy is a condition in which high blood sugar causes retinal blood vessels to swell and leak blood. Diabetic retinopathy is classified as either nonproliferative (background) or proliferative. Nonproliferative retinopathy is the early stage, where small retinal blood vessels break and leak. In proliferative retinopathy, new blood vessels grow abnormally within the retina. This new growth can cause scarring or retinal detachment, which can lead to vision loss. The new blood vessels may also grow or bleed into the vitreous humor, the transparent gel filling the eyeball in front of the retina. Proliferative retinopathy is much more serious than the nonproliferative form and can lead to total blindness.

The following articles provide background information relating to diabetic retinopathy: Spranger, J. and Pfeiffer, A. F., New concepts in pathogenesis and treatment of diabetic retinopathy, Exp. Clin. Endocrinol. Diabetes 109 Suppl. 2:S438-50 (2001), Lieth, E., Retinal neurodegeneration: early pathology in diabetes, Clin. Experiment Ophthalmol.Feb;28(1):3-8 (2000), and Wong, J. S., and Aiello, L. P., Diabetic retinopathy, Ann Acad Med Singapore Nov;29(6):745-52 (2000).

### BACKGROUND INFORMATION

The following articles provide background information relating to the invention: Felsenfeld, G., et al., Insulators and Boundaries: Versatile Regulatory Elements in the Eukaryotic Genome, Science, 291:447-450 (2001); Zhou, J., et al., Characterization of the transvection mediating region of the Abdominal-B locus in Drosophila, Development, 126:3057-3065 (1999); Zhou, J., et al., The Fab-7 element of the bithorax complex attenuates enhancer-promoter interactions in the Drosophila embryo, Genes and Devel., 10:3195-3201 (1996); Foley, K. P. and Engel, J. D., Individual stage selector element mutations lead to reciprocal changes in β-versus ε-globin gene transcription: genetic confirmation of promoter competition during globin gene switching, Genes Dev., 6:730-744 (1992); Geyer, P. K., The role of insulator elements in defining domains of gene expression, Curr. Opin. Genet. Dev., 7:242-248 (1997); Li, X. and Noll, M., Compatibility between enhancers and promoters determines the transcriptional specificity of gooseberry and gooseberry neuro in the Dropsophila embryo, EMBO J., 13:400-406 (1994); Mihaly, J., et al., Chromatin domain boundaries in the Bithorax complex, Cell. Mol. Life Sci., 54:60-70 (1998); Ohtsuki, S., et al., Different core promoters possess distinct regulatory activities in the Drosophila embryo, Genes Dev., 12:547-556 (1998); and Udvardy, A., Dividing the empire: boundary chromatin elements delimit the territory of enhancers, EMBO J., 18:1-8 (1999). The properties of enhancers are described in the following articles: Palla, F., et al., Enhancer blocking activity located near the 3′ end of the sea urchin early H2A histone gene, P.N.A.S. USA 94:2272-2277 (1997); Scott, K. C., Enhancer blocking by the Drosophlia gypsy insulator depends upon insulator anatomy and enhancer strength, Genetics 153:787-798 (1999); and Pamell, T. J. and Geyer, P. K., Differences in insulator properties revealed by enhancer blocking assays on episomes, EMBO J. 19(21):5864-5874 (2000).

### SUMMARY OF THE INVENTION

One embodiment of the invention is an isolated nucleic acid comprising a region that codes for a first regulatory module operably linked to a region that codes for an insulator, said region that codes for an insulator being operably linked to a region that codes for a second regulatory module that is different from said region that codes for the first regulatory DNA module.

A second embodiment of the invention is a method for constructing a regulatory sequence, which comprises:

a) operably linking a first sequence comprising the coding sequence for a first regulatory module with a second sequence comprising the coding sequence for an insulator;

b) operably linking said second sequence with a third sequence comprising the coding sequence for a second regulatory module, wherein said first and third sequences code for different regulatory modules.

Another embodiment of the invention is a library of isolated nucleic acids each comprising a region that codes for a first regulatory module operably linked to a region that codes for an insulator, said region that codes for an insulator being operably linked to a region that codes for a second regulatory module that is different from said first regulatory module.

Yet another embodiment of the invention is an isolated nucleic acid comprising a region that encodes a first reporter gene operably linked to a region that codes for a first regulatory module, said first regulatory module being operably linked to a region that codes for an insulator, said region that codes for an insulator being operably linked to a region that codes for a second regulatory module that is different from said first regulatory module, wherein said second regulatory module is operably linked to a region that encodes a second reporter gene that is different from said first reporter gene.

Another embodiment of the invention is a method for identifying at least one compound that interacts with a test pathway comprising:

a) providing a cell comprising an isolated nucleic acid comprising the coding region for a first reporter gene operably linked to a first control module, said first first control module being operably linked to a first regulatory module, said first regulatory module being operably linked to an insulator sequence, said insulator sequence being operably linked to a second regulatory module different than said first regulatory module, said second regulatory module being operably linked to a second control module, said second control module being operably linked to the coding region for a second reporter gene different than said first reporter gene,

b) contacting the cell with at least one compound;

c) monitoring the differences in the expression levels of the reporter genes, wherein said first reporter gene is operably linked to a control pathway and said second reporter gene is operably linked to a test pathway; whereby

d) a difference in the expression levels of the reporter genes identifies a compound that interacts with the test pathway.

Another embodiment is an isolated nucleic acid comprising the coding region for a first fluorescent protein operably linked to a first promoter sequence, said first promoter sequence being operably linked to a first enhancer sequence, said first enhancer sequence being operably linked to a cHS4 insulator sequence, said cHS4 insulator sequence being operably linked to a second enhancer sequence, said second enhancer sequence being operably linked to a second promoter sequence, said second promoter sequence being operably linked to the coding region for a second fluorescent protein, wherein the coding region for said first fluorescent protein is different from the coding region for said second fluorescent protein, and said first enhancer sequence is different from said second enhancer sequence.

Yet another embodiment is an isolated nucleic acid comprising the coding region for a first fluorescent protein operably linked to a first promoter sequence, said first promoter sequence being operably linked to a first enhancer sequence, said first enhancer sequence being operably linked to a cHS4 insulator sequence, said cHS4 insulator sequence being operably linked to a second enhancer sequence different from said first enhancer sequence, said second enhancer sequence being operably linked to a second promoter sequence, said second promoter sequence being operably linked to the coding region for a second fluorescent protein different from the coding region for said first fluorescent protein, wherein said first or second enhancer sequence is optional.

Another embodiment of the invention is a method for altering a protein-protein interaction in a test pathway comprising:

a) providing a cell comprising an isolated nucleic acid comprising the coding region for a first reporter gene operably linked to a first promoter sequence, said first promoter sequence being operably linked to a first enhancer sequence, said first enhancer sequence being operably linked to an insulator sequence, said insulator sequence being operably linked to a second enhancer sequence, said second enhancer sequence being operably linked to a second promoter sequence, said second promoter sequence being operably linked to the coding region for a second reporter gene, wherein the coding region for said first reporter gene is different from the coding region for said second reporter gene, and said first enhancer sequence is different from said second enhancer sequence;

b) contacting the cell with at least one compound;

c) monitoring differences in expression levels of the reporter genes wherein said first reporter gene is operably linked to a control pathway and said second reporter gene is operably linked to a test pathway; whereby

d) a difference in expression levels of the reporter genes identifies a compound that alters a protein-protein interaction in the test pathway.

Another embodiment of the invention is a method for affecting a compound-protein interaction in a test pathway comprising:

a) providing a cell comprising an isolated nucleic acid comprising the coding region for a first reporter gene operably linked to a first promoter sequence, said first promoter sequence being operably linked to a first enhancer sequence, said first enhancer sequence being operably linked to an insulator sequence, said insulator sequence being operably linked to a second enhancer sequence, said second enhancer sequence being operably linked to a second promoter sequence, said second promoter sequence being operably linked to the coding region for a second reporter gene, wherein the coding region for said first reporter gene is different from the coding region for said second reporter gene, and said first enhancer sequence is different from said second enhancer sequence;

b) contacting the cell with at least one compound;

c) monitoring differences in expression levels of the reporter genes wherein said first reporter gene is operably linked to a control pathway and said second reporter gene is operably linked to a test pathway; whereby

d) a difference in expression levels of the reporter genes identifies a compound that alters a compound-protein interaction in a test pathway.

Another embodiment of the invention is an isolated nucleic acid comprising a region that codes for a first regulatory module, said first regulatory module being operably linked to a region that encodes a first reporter gene, said first reporter gene being operably linked to a region that codes for an insulator, said insulator being operably linked to a region that codes for a second regulatory module, wherein said second regulatory module is different from said first regulatory module, and said second regulatory module is operably linked to a region that encodes a second reporter gene that is different from said filrst reporter gene, and said second reporter gene is linked to a region that codes for an second insulator, said second insulator being different from or the same as said first insulator.

Another embodiment of the invention is an isolated nucleic acid comprising the nucleotide sequence of SEQ ID 1 or any variant thereof.

Another embodiment of the invention is an isolated nucleic acid comprising the nucleotide sequence of SEQ ID 2 or any variant thereof.

Another embodiment of the invention is an isolated nucleic acid comprising the nucleotide sequence of SEQ ID 3 or any variant thereof.

Another embodiment of the invention is an isolated nucleic acid comprising the nucleotide sequence of SEQ ID 4 or any variant thereof.

Another embodiment of the invention is an isolated nucleic acid comprising the nucleotide sequence of SEQ ID 5 or any variant thereof.

Another embodiment of the invention is an isolated nucleic acid comprising the nucleotide sequence of SEQ ID 6 or any variant thereof.

Another embodiment of the invention is an isolated nucleic acid comprising the nucleotide sequence of SEQ ID 7 or any variant thereof.

Another embodiment of the invention is an isolated nucleic acid comprising the nucleotide sequence of SEQ ID 8 or any variant thereof.

Another embodiment of the invention is an isolated nucleic acid comprising the nucleotide sequence of SEQ ID 9 or any variant thereof.

### BRIEF DESCRIPTION OF THE DRAWINGS

FIG. 1 is a schematic representation of a regulatory assay wherein players in the control and target pathways are represented by geometric shapes. Each grouping of shapes relates to a step in the pathway. Arrows designate the next step in the pathway. A nucleic acid construct is shown comprising various elements.

FIG. 2 is a schematic representation of a regulatory assay wherein both the control and target pathways are functioning properly. Each grouping of shapes relates to a step in the pathway. Arrows designate the next step in the pathway. A nucleic acid construct is shown comprising various elements.

FIG. 3 is a schematic representation of a regulatory assay wherein a chemical compound interacts with a player in the target pathway resulting in a decreased level of transcription. Each grouping of shapes relates to a step in the pathway. Arrows designate the next step in the pathway. A nucleic acid construct is shown comprising various elements.

FIG. 4 is a schematic representation of a regulatory assay wherein a chemical compound interacts with a player in the target pathway resulting in an increased level of transcription. Each grouping of shapes relates to a step in the pathway. Arrows designate the next step in the pathway. A nucleic acid construct is shown comprising various elements.

FIG. 5 is a schematic representation of a generalized signal transduction pathway.

FIG. 6 is a schematic representation of an exemplary cell with its many signalling pathways.

### DETAILED DESCRIPTION OF THE INVENTION

The invention is composed of singular or multiple reporter gene and regulatory module combinations. The area between each regulatory module driven reporter gene is demarcated by an insulator sequence or any sequence that functions to limit the influence of the enhancer module to specific reporter genes. The insulator sequence may include insulator sequences, such as scs, scs′, fab7, fab8, the gypsy Su(Hw) array, the cHS4 region from the chick globulin locus, the BEAD element, or any other sequence with insulator properties. Each reporter gene (for example, lacZ, luciferase, GFP or GFP derivatives, alkaline phosphatase, or any other detectable enzymatic activity, binding activity or detectable RNA transcript) is attached to a control module, which contains the minimal required cis-elements that denote the region around a transcriptional start site (for example, TATA boxes, initiator elements, down-stream promoter elements, and CpG islands) but which is insufficient to maintain enhanced gene transcription.

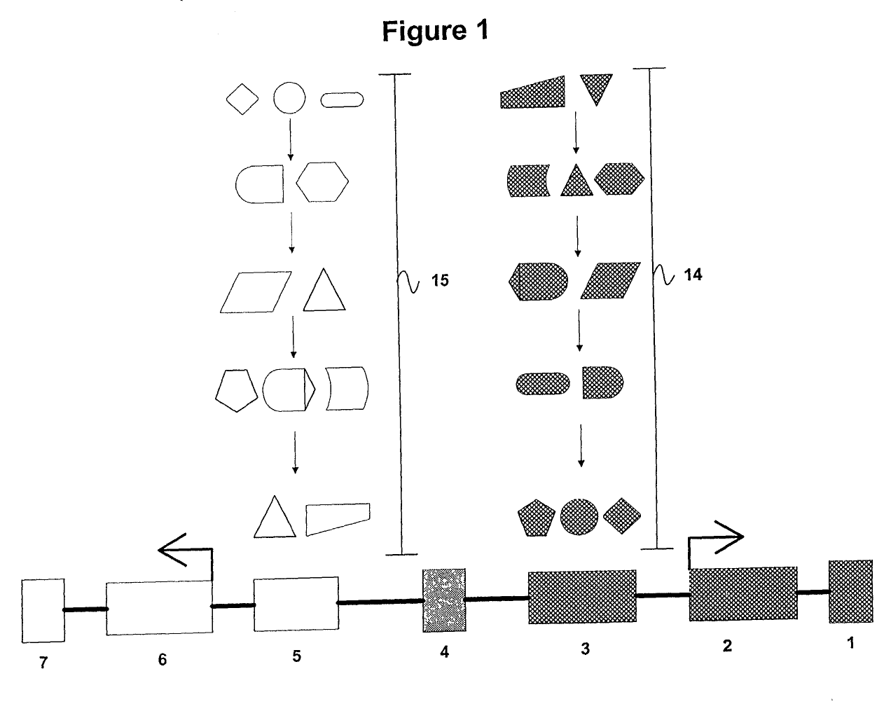

FIG. 1 is a diagram of one embodiment of the invention. A test regulatory module 3, for example, a target enhancer, which integrates the signaling activity of a set of interacting proteins known as the test pathway, as shown by 14, is shown adjacent to a basal promoter 2 and a reporter gene 1. A similar arrangement with a different enhancer module 5, basal promoter 6, and reporter gene 7 are located within the same construct. This latter reporter combination (5, 6, and 7) is known as the control reporter system while the former combination (1, 2, and 3) is known as the test reporter system. The set of interacting proteins known as the control pathway, are shown by 15. The invention provides for an insulator sequence 4, which prevents the regulatory activity in the control system from affecting the activity in the test system and visa versa. Any number of test and control systems may be similarly arranged in one construct. For the regulatory assay to function in an interpretable manner each reporter gene and enhancer module must be different from others in the construct, although the insulator and basal promoter sequences may be the same.

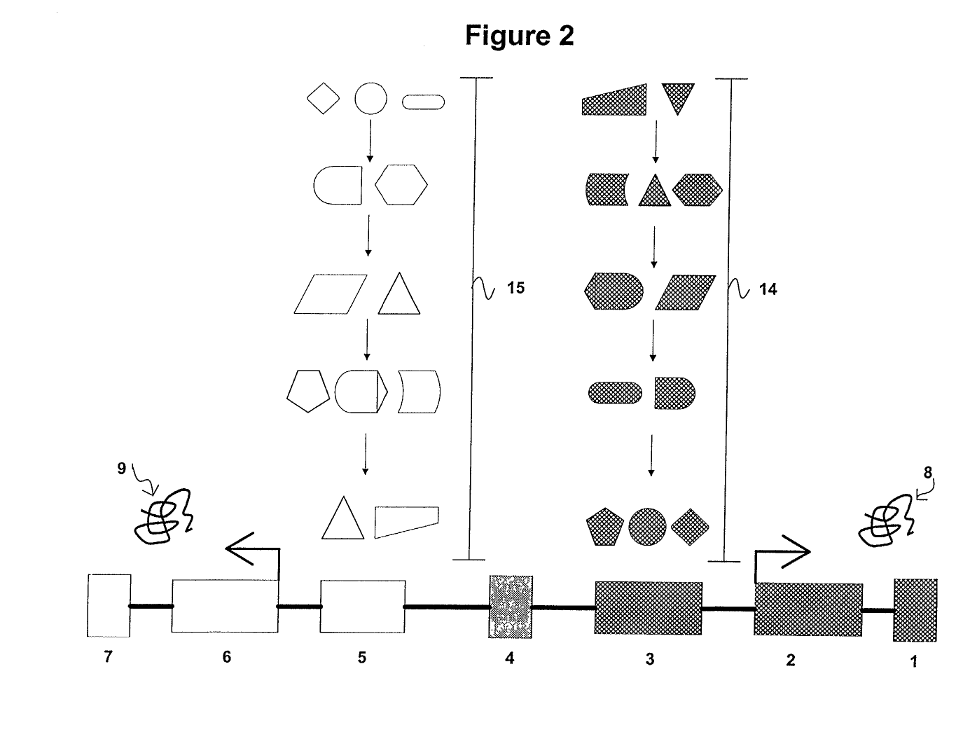

FIG. 2 is a diagram of another embodiment of the invention. A test regulatory module 3, for example, a target enhancer, which integrates the signaling activity of a set of interacting proteins known as the test pathway, as shown by 14, is shown adjacent to a basal promoter 2 and a reporter gene 1. The interaction of the test pathway 14 with the enhancer 3 results in a normal level of transcription of the reporter gene 1, as shown by protein 8. A similar arrangement with a different enhancer module 5, basal promoter 6, and reporter gene 7 are located within the same construct. This latter reporter combination (5, 6, and 7) is known as the control reporter system while the former combination (1, 2, and 3) is known as the test reporter system. The set of interacting proteins known as the control pathway, are shown by 15. The interaction of the control pathway 15 with the enhancer 5 results in a normal level of transcription of the reporter gene 7, as shown by protein 9. The insulator sequence 4, prevents the regulatory activity in the control system from affecting the activity in the test system and visa versa. For example, module 1 may encode for the expression of a green fluorescent protein (GFP) and module 7 may encode for the expression of a red fluorescent protein (RFP). The readout from a photon detector for the GFP may be 100 Units and the readout from the RFP may be 100 Units, indicating that both systems are working properly. This is an example of a control experiment for a regulatory assay. The Units are chosen based on experimental design.

Any number of test and control systems may be similarly arranged in one construct. For the regulatory assay to function in an interpretable manner each reporter gene and enhancer module must be different from others in the construct, although the insulator and basal promoter sequences may be the same.

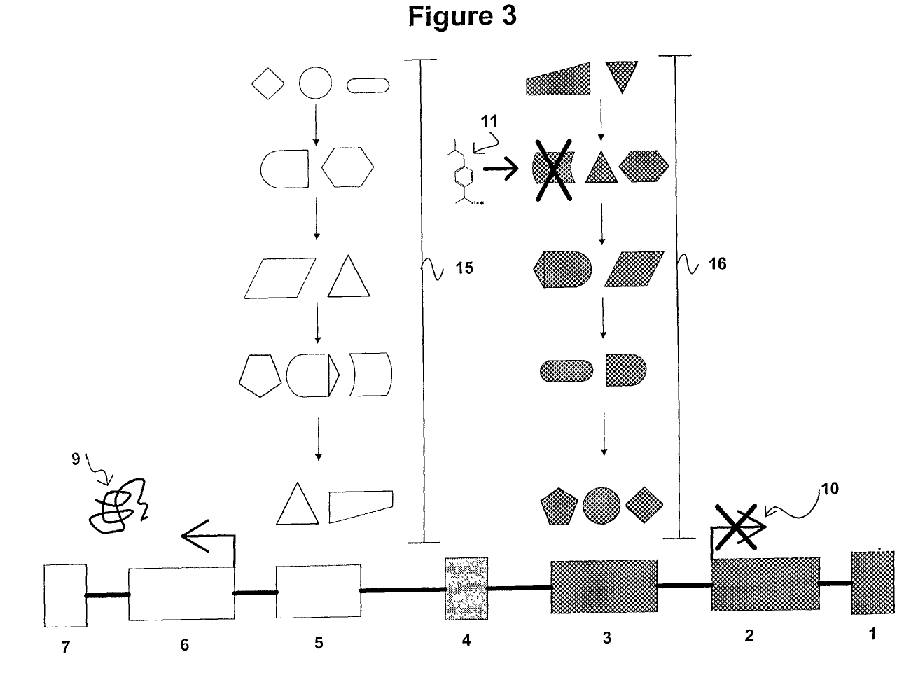

FIG. 3 is a diagram of another embodiment of the invention. A test regulatory module 3, for example, a target enhancer, which integrates the signaling activity of a set of interacting proteins known as the test pathway, as shown by 16, is shown adjacent to a basal promoter 2 and a reporter gene 1. A compound 11 is shown interacting with one of the players of the test pathway 16. The interaction of the compound 11 with the test pathway 16 results in a decrease in transcription of the reporter gene 1, as shown by 10. A similar arrangement with a different enhancer module 5, basal promoter 6, and reporter gene 7, are located within the same construct. This latter reporter combination (5, 6, and 7) is known as the control reporter system while the former combination (1, 2, and 3) is known as the test reporter system. The set of interacting proteins known as the control pathway, are shown by 15. The interaction of the control pathway 15 with the enhancer 5 results in a normal level of transcription of the reporter gene 7, as shown by protein 9. The insulator sequence 4, prevents the regulatory activity in the control system from affecting the activity in the test system and visa versa. For example, module 1 may encode for the expression of a green fluorescent protein and module 7 may encode for the expression of a red fluorescent protein. The readout from a photon detector for the GFP may be 10 Units and the readout from the RFP may be 100 Units, indicating that the control system is working, as shown by protein 9, but that the test system is affected by compound 11.

Any number of test and control systems may be similarly arranged in one construct. For the regulatory assay to function in an interpretable manner each reporter gene and enhancer module must be different from others in the construct, although the insulator and basal promoter sequences may be the same.

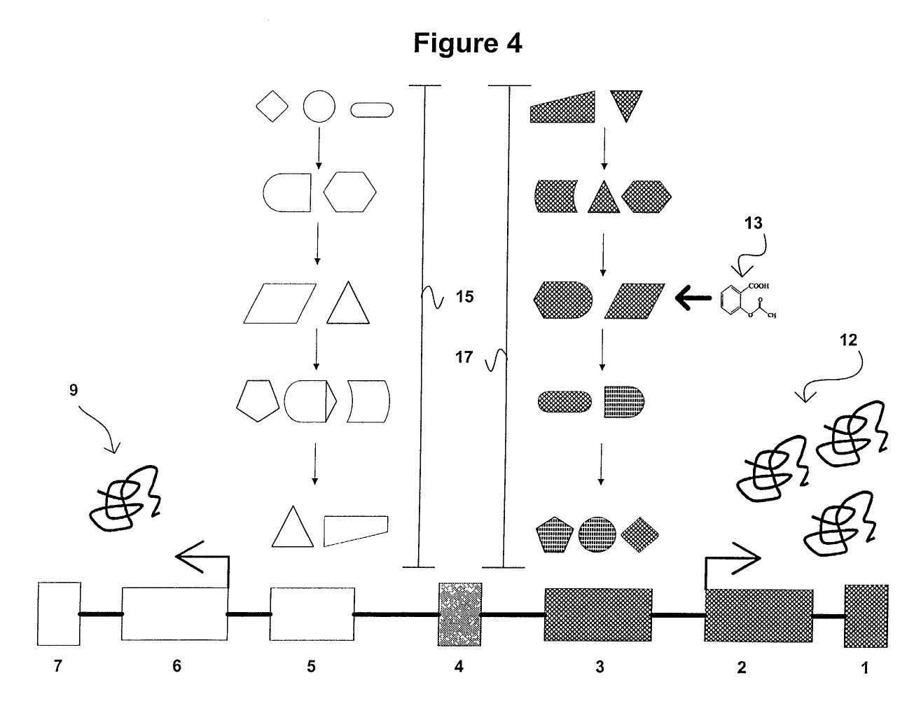

FIG. 4 is a diagram of another embodiment of the invention. A test regulatory module 3, for example, a target enhancer, which integrates the signaling activity of a set of interacting proteins known as the test pathway, as shown by 17, is shown adjacent to a basal promoter 2 and a reporter gene 1. A compound 13 is shown interacting with one of the players of the test pathway 17. The interaction of the compound 13 with the test pathway 17 results in an increase in transcription of the reporter gene 1, as shown by protein 12. A similar arrangement with a different enhancer module 5, basal promoter 6, and reporter gene 7, are located within the same construct. This latter reporter combination (5, 6, and 7) is known as the control reporter system while the former combination (1, 2, and 3) is known as the test reporter system. The set of interacting proteins known as the control pathway, are shown by 15. The interaction of the control pathway 15 with the enhancer 5 results in a normal level of transcription of the reporter gene 7, as shown by protein 9. The insulator sequence 4, prevents the regulatory activity in the control system from affecting the activity in the test system and visa versa. For example, module 1 may encode for the expression of a green fluorescent protein and module 7 may encode for the expression of a red fluorescent protein. The readout from a photon detector for the GFP may be 300 Units and the readout from the RFP may be 100 Units, indicating that the control system is working but that the test system is affected by compound 13.

Any number of test and control systems may be similarly arranged in one construct. For the regulatory assay to function in an interpretable manner each reporter gene and enhancer module must be different from others in the construct, although the insulator and basal promoter sequences may be the same.

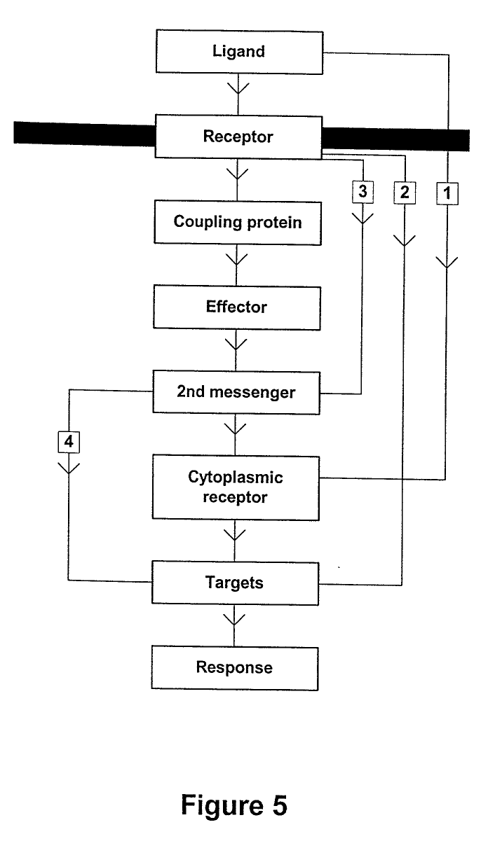

FIG. 5 provides an example of the type of control or test pathway that can be used in the invention. This pathway serves as an example of the possible complexity of a test or control pathway used by the present invention. FIG. 5 illustrates a simplified linear pathway from the recognition of an extracellular signal (ligand-receptor interaction) to an intracellular response. The steps illustrated essentially follow those employed in the β-adrenergic regulation of phosphorylase b. Also indicated in FIG. 5 are alternative strategies that can be employed following other types of ligand-receptor interactions; these by-pass certain steps. For example (i) steroid hormones are membrane permeant and interact directly with intracellular receptors [pathway 1]; (ii) there is evidence that certain growth factor receptors can directly regulate intracellular proteins [pathway 2]; (iii) some receptor have intrinsic “effector” capacity, i.e., directly produce second messengers [pathway 3]; (iv) certain second messengers act pleiotropically and interact with a number of target proteins to produce a coherent integrated response [pathway 4].

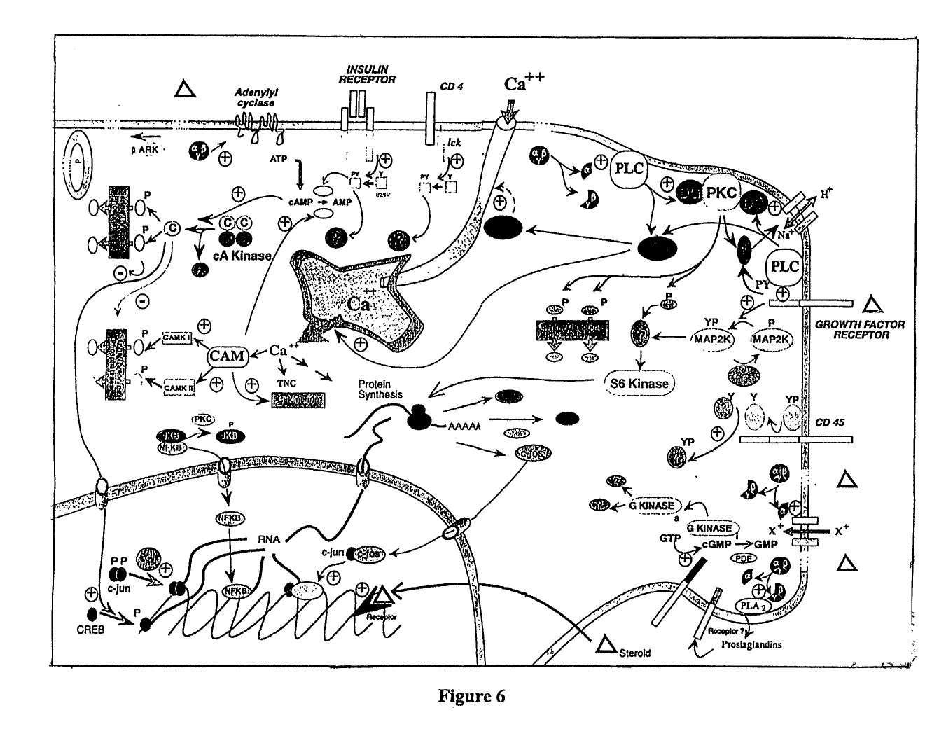

FIG. 6 provides an example of a typical cell with its many signalling pathways. The players in the pathways comprise receptors, phosphorylation events, G proteins, second messengers, hormones, growth factors, effectors, kinases, dephosphorylation events, serine and threonine phosphatases, and many others. The pathways that are exemplified are the insulin signalling pathway, the adenyl cyclase pathway, the growth factor receptor pathway, the MAP kinase pathway, and several others. Also shown in the figure are nuclear transcription factors, such as NFKB, c-jun, and c-fos.

Any of the constructs describe above can be placed within a cellular environment, such as within a tissue cultured cell line, or within an acellular environment, such as a cell extract. In both environments the protein components that compose the control and test pathways are present or expressed. The combination of the construct within the cellular or acellular context is the regulatory assay.

Libraries can be screened, such as chemical libraries or individual chemical compounds, by monitoring the relative levels of expression between the separate reporter genes. The assay functions by identifying compounds that interact specifically with any single protein component or components of a test pathway that thereby is functionally altered and is detectable by a change in the transcriptional rate as detected through the reporter gene. The control reporter system serves to control against compounds that trivially affect other transcriptional pathways.

The assay can also be used to screen for compounds that affect particular components of the pathway within either a reconstituted cellular-mimicking environment or an approximate tissue-cultured cell line, wherein only a portion of the pathway may be available. Under this condition, the environment may still be sufficient for maintaining expression downstream of the pathway and thus still falls under the claim of this invention. For example, when screening for components downstream of activated ras or activated rho, one may use a tissue cultured cell line that only contains a transfected copy of a mutated Ras or Rho GTPase gene, which is constitutively active and that also contains, or expresses, the proteins downstream of the activated GTPase. This assay serves to screen the downstream components of the RAS pathway even though the upstream components may not be present.

The invention can be used to determine the effect of various compounds on very simple to very complex pathways. For simplicity, one pathway is shown in the Figures to converge on an enhancer. Several different pathways can converge on an enhancer.

The practice of the present invention will employ, unless otherwise indicated, conventional techniques of cell biology, cell culture, molecular biology, microbiology, recombinant, which are within the skill of one skilled in the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195: Nucleic Acid Hybridization (B. D. Hames & S. J. Higgins eds. 1984); Transcription And Translation (B. D. Hames & S. J. Higgins eds. 1984); Culture Of Animal Cells (R. I. Freshney. Alan R. Liss, Inc., 1987); Immobilized Cell And Enzymes (IRL, Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Other features and advantages of the invention will be apparent from the following detailed description, and from the claims.

Isolated Nucleic Acid

The term “isolated” nucleic acid is used herein with respect to nucleic acids, such as DNA or RNA, and refers to molecules separated from other DNAs, or RNAs, respectively, that are present in the natural source of the macromolecule. In addition, an “isolated nucleic acid” is meant to include nucleic acid fragments which are not naturally occurring as fragments and would not be found in the natural state.

Nucleic acid as defined herein includes deoxynucleic acid (DNA), ribonucleic acid (RNA), and peptide nucleic acid (PNA). Chemically, PNA is based on a peptide-like 2-aminoethylglycine backbone to which the bases of nucleic acids (nucleobases) are attached by means of a carboxymethyl linker to the central secondary amine. PNA is further described in the following articles: Ørum, H., et al., Peptide Nucleic Acid, pp. 29-48, Biotechniques Books, Div. Eaton Publishing, Birkhäuser Boston, 1997; Nielsen, P. E., Applications of peptide nucleic acids, Current Opinion in Biotechnology 10:71-75 (1999); and Nielsen, P. E., Peptide nucleic acid (PNA). From DNA recognition to antisense and DNA structure, Biophysical Chemistry 68:103-108 (1997).

A variant of a nucleic acid sequence can be a portion of the sequence or a larger sequence that contains the desired sequence. The variant can have changes in the sequence as long as the sequence retains its desired function. For example, if the sequence is an enhancer sequence, the enhancer sequence can have mutations in it as long as it still performs its desired function as an enhancer. Mutations can be point mutations, internal insertions, internal deletions, truncations from either end of the sequence, and combinations thereof.

The above described variants are routine for one skilled in the art to make, and/or to detect. In addition, it would be routine for one skilled in the art to assay for the function of the enhancer using known assays.

Regulatory Modules

A regulatory module can comprise any collection of cis-regulatory sequences that compose the functional unit referred to as the regulatory module. The regulatory module can consist of an enhancer, silencer, scaffold-attachment region, negative regulatory element, transcriptional initiation site, regulatory protein binding site, any combination or multiplicity of said sequences, and any other regulatory sequence which has a transcription-rate modifying function when placed adjacent to a reporter gene. Such regulatory sequences are described in Goeddel; Gene Expression Technology: Methods in Enzymology 185. Academic Press, San Diego, Calif. (1990).

An enhancer is an sequence that is present in the genomes of higher eukaryotes and various animal viruses, which can increase the transcription of genes into messenger RNA. Enhancers are often found 5′ to the start site of a gene, and when bound by a specific transcription factor, enhance the levels of expression of the gene, but are not sufficient alone to cause expression. Enhancers can function in either orientation and at various distances from a promoter.

Examples of enhancer containing sequences are EPO 3′ hypoxia enhancer, cytoplasmic actin promoter, VEGF hypoxia enhancer, LBP-32 enhancer, relA hypoxia enhancer, PROC hypoxia enhancer, DELTEX hypoxia enhancer, HMOX1 enhancer, GRAP enhancer, BTEγ-4 hypoxia enhancer, CCRdelta5 lymphocyte promoter, and COL4A1 . This list is merely an exemplary list of the types of enhancers that can be used in the present invention.

Examples of several enhancer sequences are provided below:

The sequence for the HMOX1 enhancer (SEQ ID 1):

ccacccgccccccccccccccgcccaggcgtacccccccttaccccgcc
ccccacccgctcgccgcgcccagcccatctggcgccgctctgcccctgc
tgagtaatcctttcccgagccacgtggccgtgtttttcctgctgagtca
cggtcccgaggtctattttcgctaagtcaccgccccgagatctgttttc
gctgagtcacggtcccggtgtctgttttcgctgagtcacggtctagaga
tttgttttcctcagagttccagctgctccaggtttaatcccctggggca
aagtccggactgtccggctggagtctggagtcgggacatgcctcagcca
gcacgtcctcggcctcgtctggggcctgaatcctagggaagccatagca
gctcctccacccttcctctcactcctcctctagcctcttgctactcccc
gcaccactgttttagggaacctctatctcccgacggcctgccacgggcc
aggcgctgtgctgggggcttcacactttaaatcgctgttgagcggggcg
cgggggcgctgcaacctaaaggtgggagctactcaaatggaggggcatc
tgttaaaatggccggcctgtcattttcaaaaacttcaaggccgggcgcg
gtggctcacgcctgtaatcccagcactttgggaggccgaggcgggcgga
tcacgaggtcaggagatcgagatcatcttgtcta

The sequence for the BTEB hypoxia enhancer (SEQ ID 2):

ggccggccgcttccgcacccctccaaccccggccacgtggggatcggag
ccctcctccatcgtgaacgggggcgcgcccggggccaggagggaagcat
ccggggagggggcgggctcccgccggccgcgcgccccggcgtggggtgg
gcgcgccagcgaggaggcggggcgcgctctgggagggaggggcgtgccg
ggatggggcggggcgcgctctcggaaggaggggcgtgccggagaggggg
cggggtcctcggcctcccgccccggcttgagggggtggtgctccgggaa
agggtacgggggcgcgcgctggagtggggcgcgccgggagggggcgggg
ctcccggccttgcaccctagcgcggagaaggcgcgctccgggcctaaga
cgctaggcgctggcggaggccggagtgggcgcgccggggggcgggttcc
tccggcccgagccccgcccccggcggccgcgcgcggctcggagagtggt
ggagggcgcgccgggcggggggcgtggctgcggcgccccgagggggcgt
cctcc

The sequence for the CG Orphan H1 enhancer (SEQ ID 3):

ggggaggggcggcgggggcggggccgggaactcaggtgggcgtgggaag
gacggggctggggctggggctgggaagatgaggtgggggcactggactg
ggatgggaagaaagtaagggatcggaacagcggtgagggagcggtgggc
cacgtcccagggctcagcgtgcctctacgtgcagggaacccatatccca
gatttccggagctgcctgaagtcctcgcaacttctgagggaaactaggg
cagccggggaacttcccagtagcttcttagagtgggaggcggccccggc
acagagtcgccccgcaaaccgagggcttccgggtaagggaggggtctta
aaatttccgggtgccggcaacccaggaggcctgcccggaggaggctggg
gctctgggaggggtccaggagacccagaaacgcg

The sequence for the Col4A1 hypoxia enhancer (SEQ ID 4):

cctaggtacatttggtgcggaacttgccccaagcagcaagtcgtgggga
aatgtgaaaccagcaagaactgcccccagggaaattgaagataaaacac
aaaaccgaatttaaaaagtcacctgctgctccatttcaaactggaagtc
taaaaaaggcatttcctgacgctggcgagtgactcagtgtcaccgtgac
tcagccgcgcccggttgcccacgaggcgggagggggaggcagatgctgc
gggcggcgcggggagccgagcccgcgcgctgctatttcgggcaagtctg
cggcgagcagggcccgcagtccacgcgcactcaggaagtacaaataggg
cgtgcatcagaggaagcgctccccacgcagaggctgtgggaaagtgatt
cagcgctgctagaatccccctctcccgcgcccttcgcagcgcagccagg
gagggagggagcgcgccagagcctcctgcaggtgcgcgcggggcaggcg
ggcgggcgggcggcgcgcgcgcccagggggcccaggtgaccgccttccg
cgcgcacacagccggccagggcgcacccggaagcccgttacccaaaggc
aaggcttgttccagcctaaagcaaactcatccacacaacgtcgctagcg
agttccgtttccctgtttctgggaatttgtgactgtcaa

The sequence for the Deltex hypoxia enhancer (SEQ ID 5):

cagccaccaagtgggtgtgtgtgcactgcacctgtgtgcatgtgtgatt
gtgcctctgcatgtgtgcaggtgtgggcctcccaggctgagtgtctgtg
tgtcagtgtcaggaggtgtgggtcagagcatgtgcgcatgtgtcctcgt
gtgcagtgagtcagcgtgtcctcctgcgcccccatgcatgatgtgaatc
agtacgcacgagtacttccccacaggcggtgcggaatcgtgtgtccgcg
tgtggatggcaggccattctgcaggctgcagcgcgccgggggcttgtag
ggctgagcgcatgctcctctgggtttgctgcccactttacccagctctg
tgcagctggggagaggggcggcagtggcaggtgcggtactggtctacat
gagccaccgactcttgtccaatggagtatcctcccaggggagagctgtg
tgtgcttctgtgtgtatgtgtgtgtccccgtgtgtgcacaagtctctgt
atgtaggtgt

The sequence for the GRAP hypoxia enhancer (SEQ ID 6):

gggagaacactgtccctctctgggatgggtgttccaggagctcttgggc
ttaggcctctgatattttcggaattcgggcaccaggggaccgtgggcag
tgcgtccgccccaggtctgtctgtctgtcgaggggtgaatcagcttccg
gccccgccccgggcggcacgtgaccgcaggtggcggcggcggggtaagc
ggggcggccctgagtcaccggcgcgcccccgcccagcctcgcgccgccg
ccgcagccgccgcgtgtgcgccccgctccgccagcgcccgctcggtaag
caagtcccggccgcgccccccggatcccgggtctcgcggggcgtcgccg
ccccgcaaccgcgtttctgggtctcctggacccctcccagagccccagc
ctcctccgggcaggcctcctgggttgccggcacccggaaattttaagac
ccctcccttacccggaagccctcggtttgcggggcgcctctgtgccggg
gccgcctcccactctaagaagctactgggaagttccccggctgccctag
tttccctcagaagttgcgaggacttcaggcagctccggaaatctgggat
atgggttccctgcacgtagaggcacgctgagccctgggacgtggcccac
cgctccctcaccgctgttccgatcccttactttcttcccatcccagtcc
agtgccc

The sequence for the PROC hypoxia enhancer (SEQ ID 7):

ctgactcacaggctgactcagctgcaggcgcgctgccaggcgacgcagc
gggcgggtggccgggcgccggcgggctcgcagccgggctgctggcaacg
gtgccggcggaggtgggggcgtggcgcgggatgggcggcgcgggccctg
ccgtggtaccgcctggcagcgtccaccccgccgctggggcgccctggag
gctcctggccctccgtggggccgtgacaccggcgctgcggggagcggtg
gcctcgcagaggctgggcatgggaggacggccgccccgggtaaaggaca
gggccctggaaacgcgggtctgccgggagcaggggacaggaaggagacc
gcggctctcccagtcctgctgccccgggcctccagacggccagactctc
cccacaccggcctggagggggacgcgccgaccccagctgggaggggtgg
ctggctgcgtagatccgtttgggccgcctgcctggaaaggcccaggtcc
gggctcgtcc

The sequence for the relA hypoxia enhancer (SEQ ID 8):

gaaaggggagggagatcagggtcagcacacacccaatgcccattctcac
aaggaggaatctgctctccacggagaggagaagccaggctccctcccag
gggaactgagtcaggacctgctccctagaacctgcggtgaaactaaggg
gtggaggaaaggaaccagaaacacctgcttcttgagggaaaacggggta
aggaatccttcttctccagagggaagctgaatcagggcctgttgtactt
tcttaaggaaaattgagggagggcacgccccacctccctccagagagga
aactgaatcagatgcgttctcccctaatagggaaacgaagccagagctg
cccccatggacggtgtggagggaaactgagtcaaggttccctctgctcc
cccacccagggaggatgctgagtcaagggccaccccctccacccagagg
ggaaactgagtcagacccctccccgcctgccccgccccgcgccaccatc
cggcaggccgaccgctccctgcgcagctccgtcgacgggaatggggcgg
aaacgcggcgcgtgcggctccgcacagccgtgcggccccggcgattgca
ccccgcggggtcagagggcgacctcaccgtccatggccggggtctcggg
gcgtggcggggtcgcagctgggcccggcgttgcactacagacgagccat
tcgccagacgancatgcgcccgcgcccgccgtcgctcactgcccggaat
ccgccgcgctcctgccccgccgccgccc

The sequence for the LBP-32 hypoxia enhancer (SEQ ID 9):

gaggttcaaggacccacttactctggtggccacttactctagaggcctg
agtacgccaggctgtgaagctctgccaagtacctgggaatctatatcca
tgcggggcaccatctcaaactgcatgagtcaggtgggcaggcgtgcata
tgggcgagtcaggtgggcaggcgtgcgtatgggcgggtcaggtggacag
gcatgtgtatgggcaggtcaggtggccaggcgtgcgtatgggcgagtga
ggtgggcaggcaagtgaggtgggcaggcgtgcatgtgggcgagtcaggt
gggcaggcgtgggtgtgggcgagtctggacaggcgtgcatacgggcggg
tcaggtgggcaggcgtttgtatgggcgggtcaggtgggcagacgtgtgt
atggtcggtttaggtggacaggcttgagtatgggtgtgcgtatggacga
gtcaggtggacaggcgtgcgtatgggagagtcaggtggacaggcgtgcg
tatgggcgagtcaggtggacaggcgtgcgtatgggcaagtcaggtggac
aggattgcgtatgggagagtcaggtggacaggcgtgcgtatgggtgtgc
gtatggacgagtcagctgggcaggcgtgcgtacaggcgggtcaggtggg
caggcgtttgtatgggcgggtcaggtgggcaggcgttcttatggtcggg
tcaggtgggcaggcgtgcatatggtcgggtcaggtggacaggcatgtgt
atgggtgtgcgtatggacgagtcaggtggacaggcgtgcgtatgggaga
gtcaggtggacaggcgtgcgtatgggagagtcaggtggacaggcgtgcg
tatgagcgagtcaggtggacaggcatgcatatgggagagtcaggtggac
aggcctgcgtatgagcaagtcaggtggacaggcgtgcctatgggtgtgc
gtatgggtgagtcaggtgggcaggcgtgcatatgggtgggtcaggtggg
caggcgtgcgtacgggcgggccaggtgggcaggcatttgtatgggccgg
tcaggtgggcaggcgttcttatggtcgggtcaggtgggcaggtgtgtgt
atgagcgagtcaggtgggcagacatgcatacgggcgggtcaggtggaca
ggcgtgtgtacgggcgggtcaggtggacagtattggcgtgcgtacgggc
gggtcagatggacaggatcgcgtacgggcgggtcaggtggacagtattg
gcgtgcatacgggcaggtcagatggacaggatcgcgtacaggcgggtca
ggtggacaggcatgcgtacaggcgggtcaggtggacaggcgtgcgtaca
ggcgggtcaggtggacaggcgtgcgtacaggcgggtcaggtgggcaggc
atgtgtgtgggagagtcaggtggacaggattgagtacgggccggtcagg
tggacaggattgcgtacgggcgggtcaggtgagcaggcgtgcgtactgg
cgggtcaggtggacaggattgcgtacgggcgggtcaggtggacaggatt
gcgtacgggcgggtcaggtgggcaggcgtgcctatggtcgggtcaggtg
ggcaggcatgcgtatgggtgagtcaggtggacaggattgcttatgggcg
ggtcaggtggacaggcgtgcgtatgggcgagtcaggtgggcaggcgtgc
gtacgagcgggtcaggtggacaggcatgcgtatgggcgggtcaggtggg
caggcgtgcgtacgagcgagtcaggtggacaggcgtgcgtatgggcggg
tcaggtgggcaggcgtgcgtatgagcgggtcaggtgggcaggcgtgcgt
atgggcgggtcaggtgggcaggcgtgcatatgggtgggtcaggtggaca
ggtgtgcgtatgagtgtgcgtatgggcgagtcaggtgggcagacgtgca
tatgagtgtgcatatgggtgattcaggtggacacgcgtgggtatgggtg
gctcacgtggacaggcgtgcgtgtcagcgtgtcacgtggacaggcgtgt
gtgtgggcgggtcaggtgggcaggcgtgtgtatgggcgggtcaggtggg
caggcgtgcatatgggcgggtcaggtgggcaggcatgcctatgggtgtg
cgtatgggcgagtcaggtgggcaggcgtgcctatgggtgagtcaggtgg
gcaggcgtgtgtatgggtgtgcatatgggcgagtcaggtggacaggcat
gcgtatgggcgggtcaggtggacaggcatgtgtatgggcgggtcaggtg
gacagacgtgcgtatgggtgagtcaggtgggcagacgtgcgtatgggtg
agtcaggtggacaggattgcgtatgggcggatcaggtggacaggcgtgc
gtatgggcgagtcaggtggacaggattgcgtatggtcgggtcaggtggg
caggcgtgtgtatgggcaggtcaggtggacaggactgaatatggggggg
tcaggtgggcaggcgtgcgtatggtcgggtcatgtgggcaggcgtgctt
atgggcaggtcaggtgggcaggtgtgcatgtgggagagtcaggtggaca
ggattgaatatgggcgggtcaggtgcgcagacatgcgtatggccgggtc
acgtgggcaggcgtgcgtttgggcgggtcaggtgggcaggggtgcatat
gggcgggtcaggtggacaggcgcacgtatgggtgagtaagatggacagg
catgtgtgtgggagagtcaggtgaacaggattgcttatgggctagtcag
gtggacaggtgtgcgtatgggtgtgcctatgggagagtcaggtggacag
gattgcgtatagccggatcaggtgggcaggcatgcgtatgagcgagtca
ggtgggcagacatgcgtatgggcgggtcaggtggacaggcgtgtgtata
ggcaggtcaggtggacaggcgtgcatatgggtatgtgtatgggcgggtc
aggtgggcaggcgtgcatacgggcgggtcaggtggacaggcgtgcatac
gggcgggtcaggtgggcaggcgtttgtatgggcgggtcaggtgggcagg
agttcttatggtcgggtcaggtgggcaggcgtgcatatggtcggtttag
gtggacaggcgtgtgtatgggtgtgcgtatggacgagtcaggtgggcag
gcatcctacaggcgggtcaggtgggcaggcgtttgtatgggtgggtcag
gtgggcaggtgttcttatggtcgggtcaggtgggcagacgtgcatatgg
tcgggtcaggtggacaggcatgtgtatgggtgtgcgtatggacgagtca
ggtggacaggcgtgcgtatgggagagtcaggtggacaggcatgtgtatg
ggcgagtcaggtggacacgcgtgcgtatgggcaagtcaggtgggcaggc
atgcgtatgggagagtcaggtggacagccctgcgtatgagcgagtcagg
tggacaggattgcgtatgggcaagtcaggtggacaggcgtgcgtatgag
tgagtcaggtggacaggattgcgtatgggagagtcaggtggacagccct
gcgtatgagcgagtcaggtggacaggattgcttatgggcaagtcaggtg
gacaggcgtgcgtatgagcgagtcaggtggacaggattgcgtatgggag
agtcaggtggacaggcgtgcatatgagcgagtcaggtggacaggcatgc
gtatgggagagtcaggtggacaggcatgcatatgagcgagtcaggtgga
caggcgtgcgtatgggtgtgcatatgggtgagtcaggtgggcaggcgtg
catacgggcgggtcaggtgggcaggcgtttgtgtgggccggtcaggtgg
gcaggtgttcttatggtcgggtcaggtggacaggcatgtgtatgggtgt
gcgtatggacgagtcaggtgggcaggcgtgcgtatgggagagttaggtg
gacaggtgtgcgtatgggtgagtcaggtggacaggcgtgcatatgggtg
tgcgtacgggcgagtcaggtgggcaggcgtgcatatgggtgtgtgtatg
ggtgagtcaggtgggcaggcatgcgtatgagcgagtcaggcgggcaggc
gtgcgtatgggtgagtcaggtgggcaggagtgcatatgggtgtgcgtat
gggtgagtcaggtgggcaggcatgcgtatgagcgagtcaggtgggcagg
cgtgcgtatgggtgagtcaggtgggcaggagtgcatatgggtgtgcgta
tgggcgagtcaggtgggcaggcgtgcgtgtgggcgagtcaggtgggcag
gcatgcatacgggcgggtcaggtgggcaggctttcttatggtcgggtca
ggtgggcaggcatgcgtacgggcaggtcaggtgggcaggcgtgcatacg
ggcaggtcaggtgtgcaggtgtttgtatgggcgggtcaggtgggcaggc
gttcgtatggtcaggtcaggtgggcaggtgttcctatggtcaggtcagg
tggacaggcgtgcgtatgggtgtgcatatgggcgagtcaggtggacagg
cgtgcgtatgggtgtgtgtatgggtgagtcaggtggacaggattgcgta
tggatgagtcatgtggacaggcgtgtgtatgggtgtgtgtatgggtgag
tcaggtgggcaggcgtgcgtatgggcgagttaggtggacaggcatgtgt
atggacgagtcagatgggcaggtgtgcgtatgggtgtgcgtatgggcga
gtcaggtggacaggattgcgtatgatcgggtcaggtgggcaggcatgtg
tatgggcaggtcaggtggacaggattgaatatgggggggtcaggtgggc
aggcgtgcgtatggtcgggtcatgtgggcaggcgtgcttatgggcgggt
ctggtggacaggattgcgtacgggcgggtcaggtgggcaggcgtgcgta
tgggcgagtcaggtggacaggattgcttatgggcgggtcaggtggacag
gcgtgcgtatgggcgggtcaggtgggcaggcgtgcgtacgagtgagtca
ggtggacaggcgtgcgtatgggcgggtcaggtgggcaggcgtgcgtatg
ggcgggtcaggtgggcaggcgtgcatatgggtgggtcaggtggacagga
ttgggtatgggtgagtcaggtggacaggcgtgcgtatgagcgggtcagg
tgagcaggcatgcgtatgggcgggtcaggtgggcaggcgtgcatatggg
tgggtcaggtggacaggtgtgcgtatgagtgtgcgtatgggcgagtcag
gtgggcagacgtgcatatgggtgtgcatatgggtgattcaggtggacag
gcgtgcgtatgggcggctcacgtggacaggcgtgcgtgtcagcgtgtca
cgtggacaggcgtgtgtgtgggcgggtcaggtgggcaggcgtgtgtatg
ggcgggtcaggtgggcaggcgtgcgtatgggcgggtcaggtgggcaggc
atgcctatgggtgtgcgtatgggcgagacaggtgggcaggcgtgcctat
gggtgagtcaggtgggcaggcgtgtgtatgggtgtgcatatgggcgagt
caggtggacaggcatgcgtatgggtgggtcaggtggacagacgtgcgta
tgggtgagtcaggtgggcagacgtgcgtatgggtgggtcaggtgggcag
gcgtgcgtatgggtgagtcaggtggacaggtgtgcgtatgggtgagtca
ggtggacaggcgtgcgtatggctgtgcgtttgggcgagtcaggtgggca
gacgtgcgtatgggcagtcaggtggacaggcgtgtgtatgggtgagtca
ggtggaccactgtgtgtacgggcgagtcaggtggaccggcgtgcatatg
ggcaggtca

The sequence for the hypoxia enhancer in EPO locus:

ctgactcagacctcacaggtggccccggtcccctgcgggccaacagctc
acctggggcatgtctacacagcagcgctgccagtggtgggtccatctgc
tcccaccataggtctatctaagtctctcagggccggcgccttagtgctg
gccctgcagccccagggagccagccaggctccttggggaagcttctcac
agtcactctctcttctcgagattggccagaagcctggagctggggccct
tccgaacgtgtccttcctgtttcccacccagccccgtggcttgttcgtc
tccttctcacccaagcttctccctccaccagcaatggcctgcccgccca
acagcaagtactccctgtgtgcgaagccatgccctgacacctgccattc
aggattctccggcatgttctgctcagaccggtgcgtggaggcctgtgat
gcaatccgggcttcgtcctcagtggcctcgagtgcatacctcgctccca
gtgtgggtg

The sequence for the hypoxia enhancer in HMOX locus:

ccgcccaggcgtacccccccttaccccgccccccacccgctcgccgcgc
ccagcccatctggcgccgctctgcccctgctgagtaatcctttcccgag
ccacgtggccgtgtttttcctgctgagtcacggtcccgaggtctatttt
cgctaagtcaccgccccgagatctgttttcgctgagtcacggtcccggt
gtctgttttcgctgagtcacggtctagagatttgttttcctcagagttc
cagctgctccaggtttaatcccctggggcaaagtccggactgtccggct
ggagtctggagtcgggacatgcctcagccagcacgtcctcggcctcgtc
tggggcctgaatcctagggaagccatagcagctcctccacccttcctct
cactcctcctctagcctcttgctactccccgcaccactgttttagggaa
cctctatctcccgacggcctgccacgggccaggcgctgtgctgggggct
tcacacttta

The sequence for the hypoxia enhancer in VEGF-B/FKBP2/PLCB3/BAD locus:

gcacaaataccagcatgcctggtcttccaagaattcggggaacccagga
gctttggcctggagtgggatctagactacagatcccagcatgccctgtg
agacacacacacacacacacacacacacacgcctggagtgggatctaga
ctacagatcccagcatgccctgtgacacacacacacacacacacacaca
cacacgcctggagtgggatctagactacagatcccagcatgccctgtga
ctgacacacacacacacacacacacacacacacacacacacacacgcct
ggagtgggatctagactacagatcccagcatgccctgtgactctctcac
acacacacacacacacacacgcctggagtgggatctagactacagatcc
cagcatgccctgtgactcacacacacacacacacacacacacacacaca
cacacacacacacgcctggagtgggatctagactacagatcccagcatg
ccctgtgactcacacacacacacacacacacacacacacacacacacac
gcctggagtgggatctagactacagatcccagcatgccctgtgactcac
acacacacacacacacacacaccaggcgggggaagagctggggagtggg
ggcggggaagtcttgtgactacaaatcccagaatgctctggagctagga
aggaacaggacggctttggggaggggtcgtgggactccagatccgggcg
tgccttgggacaaggcagggacagaccggcggggggcggcctqgtactt
caggtcctggcacgctggggactggcgctcaccttaaaggagtccacaa
actcgtcactcatcctccggagctcgcggccatagcgctgtgctgcc

Control Modules

Useful expression control modules can comprise for example, a viral LTR, such as the LTR of the Moloney murine leukemia virus, the early and late promoters of SV40, adenovirus or cytomegalovirus immediate early promoter, the lac system, the trp system, the TAC or TRC system, the T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast alpha-mating factors, the polyhedron promoter of the baculovirus system, and other sequences known to control the expression of genes of prokaryotic or eukaryotic cells or their viruses, and various combinations thereof. Suitable eukaryotic promoters are the CMV immediate early promoter, the HSV thymidine kinase promoter, the early and late SV40 promoters, the promoters of retroviral LTRs, such as those of the Rous sarcoma virus (“RSV”), and metallothionein promoters, such as the mouse metallothionein-I promoter.

Selection of appropriate vectors and promoters for propagation or expression in a host cell is a well known procedure. And the requisite techniques for vector construction, introduction of the vector into the host, and propagation or expression in the host are routine to those skilled in the art. It will be understood that numerous promoters and other control sequences not mentioned above are suitable for use in this aspect of the invention, are well known, and may be readily employed by those of skill in the art.

In addition, DNA coding for a desired product can be placed under the control of an inducible promoter, with the result that cells as produced or as introduced into an individual do not express the product but can be induced to do so. Also, a promoter can be a constitutively active promoter.

It should be noted that a control module can be located on the same vector as the regulatory module and/or on a different vector. For example, if needed the control sequence, i.e. promoter, can be “operably linked” to a regulatory module on another vector.

Insulators

Insulators mark the boundaries of chromatin domains by limiting the range of action of enhancers and silencers. Insulators, which flank many genes, may be responsible for providing a barrier against incursions from surrounding domains. Although the insulator elements vary greatly in their sequences and the specific proteins that bind to them, they have at least one of two properties related to barrier formation. First, insulators have the ability to act as a “positional enhancer blocker.” If the insulator lies between a promoter and an enhancer, then enhancer mediated activation of the promoter is impaired, but if the insulator lies outside the region between enhancer and promoter, little or no effect is observed. Insulators are neutral barriers to enhancer action; they do not inactivate either the enhancer or the promoter. Second, insulators have the ability to protect against position effects. When genes are removed from their native context, as in transgenic animals, the dominant effect of the new chromosomal environment becomes apparent. Expression levels at the new location often bear no resemblance to that of the gene in its native position. Flanking a transgene with insulators can suppress this variability. Having the ability to protect against position effects and/or to block distal enhancer activity has come to form the operational definition of an insulator. Insulators can act as a modulatable switch, allowing them to function as sophisticated regulatory elements (Bell, A. C., et al., Science, Vol. 29:447-450 (2001).

Examples of insulators that can be used in the present invention are scs, scs′, fab7, fab8, the gypsy Su(Hw) array, the cHS4 region from the chick globulin locus, VEGF-A basal promoter region, and the BEAD element. However, other sequences with insulator-like properties may also be used.

If there are multiple reporter genes contained in a nucleic acid construct and they are not separated by an insulator, crossreactivity can occur between pathways, therefore not allowing one to study the effects of a compound on a particular pathway. The insulator or a sequence with insulator-like properties is needed to be able to measure the effect of a compound on the test pathway.

The insulator of the present invention can have additional nucleic acids at both ends or at one end of the insulator sequence.

Examples of several insulator sequences are provided below:

The sequence for the chick beta-globin insulator:

gagctcacggggacagcccccccccaaagcccccagggatgtaattacg
tccctcccccgctagggggcagcagcgagccgcccggggctccgctccg
gtccggcgctccccccgcatccccgagccggcagcgtgcggggacagcc
cgggcacggggaaggtggcacgggatcgctttcctctgaacgcttctcg
ctgctctttgagcctgcagacacctgggggatacggggaaaaagcttta
ggctgaaagagagatttagaatgacagaatcatagaacngcctgggttg
caaaggagcacagtgctcatccagatccaaccccctgctatgtgcaggn
ntcatcaaccagcagcccagcgcgtcagagccacatccagcctggcctt
gaatgcctgcctgcagggatggggcatccacagcctccttgggcaacct
gttcagtgcgtcaccaccctctggggaaaaactgcctcctcatatccaa
cccaaacctcccctgtctcagtgtaaagccattcccccttgtcctatca
agggggagtttgctgtgacattgttggtctggggtgacacatgtttgcc
aattcagtgctcacggagaggcagatcttgggataaggaagtgcaggac
agcatggacgtggacatgcaggtgttgaggctctggacactccaagtca
cagcgttcagaacagccttaaggtcaagaagataggatagaaggacaaa
gagcaagttaaaacccagcatggagaggagcacaaaaaggccacagaca
ctgctggtccctgtgtctgagcctgcatgtttgatggtgtctggatgca
agcagaaggggtggaagagcttgcctggagagatacaggctgggtcgta
ggactgggacaggcagctggagaattgccatgtagatgttcatacaatc
gtcaaatcatgaaggctggaaaagnnctccaagatccccaagaccaacc
ccaacccacccaccgtgccactggccatgtccctcagtgccacatcccc
acagttcttcatcacctccagggacggtgacncncncctcctccgtggc
agctgtgccactgcagcaccgctctttggagaaggtaaatcttgctaaa
tccagcccgaccctcccctggcacaacgtaaggccattatctctcatcc
aactccaggacggagtcagtgagaatatt

The sequence for the fab-8 insulator:

ctgggttcattattttaaaactaaaaattgttctcaaataccataaact
tctacttgcagaacttgtactcttgttatcaaagcagaattcaaattta
taatgaaagttattgttataaaaatgtacgtctacatatgcttcatgta
catatatgtatgtcttataatttatgaaaaattatatcaaacgaaataa
tattaacgaaaacattttttatatatgcagacatcttccgttcatccgt
ttcaataatataaggaaagattttgaaaagagataatgacatggcacaa
tcaagttaatgttggaaatgtaattatatcttaacctttatttaaatgt
gagttgttaggatttttgaatataagaactggtgtcttaaatacactta
ttgtctgttcaatcgaaccattgaaagttatcgaacattttttacgcga
catgtccactgtcggagagcgacatcttgtgttggtgctttccttccta
gttctacattaccaaggccaggtggcgctgcaaggcgggaatgtggaaa
tttaaagtactctttctctcttcgtacttcgaagcagagaatggaactc
ttcgcttgctcaccaacacaaatgccttgcacagcttgccactgtgtga
gtgagtcttattcgaatgtgtttgtgctggttggcgttgacgtcgattt
cggatttctgctttctgagcagaaaaatgccgtaggaagcattccaaaa
cagcattcaaagtattgaagaaaattccctccaatatgcagaccaacca
gatttaggaacaagattccatcttattccataatgagctcatatagatg
acatcttatccatatactatttctcattccgaatataataatttatgga
agctttgtcaaactcacatccatttcacaaattaaaattggttgctatc
gcttgtttttccaattgatttatggcctacataaaaatttgagcttgga
aaactcgtcaaaggaaaaccgaacacgtgtgccaaactattaaaggtag
aaatttttgacggggaatataaattttgaatgagatattaattaaacaa
attgtcagccgtcgaagtcgggaggcaaaactataatttatatgccaaa
gactcaaatattactttcaagtgagacatcaggaagaggttcgttggaa
aagtcaagtagccgataaatttggatggcaacgggtctggaattctaaa
acaaaccagaaatcaaaacctcaactgattaccaattgaaattcccggt
tcaagtgcggcacttgtccacgaatctttatggattccgccgcaggaat
ggggaattcaaaaatggaaaaccgaggccatgatgcccgtttttcgata
tcaaaacatgtttgacaaatggcttctctcaattgccgatttctcaagt
gcactctgttctgagattctcttggaaggaccctcgtcagtgggcataa
ttcgcttgtcaaattaatcgagaggaagtcataaattcttaagttaatc
atattcgatgcgtacgagtaaccatctgcacttaaatatattttagaat
tttcttattatttgccacacgcattgtgactccatcttttttttaaatg
ccaaatccaggtgttatggatttacgctctctatctgatcatcacacac
ggttttctttaattaagacactgtgaaatcaataaaattaa

The sequence for the scs insulator:

gatctgcgtatgataccaaatttctgagattaagttgatatttcatttt
tttttattttattttgatggttgaaagattgatatagataaagacgtaa
attgaacttttgtttccttaacatgacaaagaaacttgcacgccaacta
aatatttgtatatattttcagttaaatcgaatcacatgctaagtatatt
aataaaacgtttgtctttgtaaaatcaacgaactgttgatcatctatcc
gtcctgttttgctcccagtgtagcggcaacagttgcactgtttggtgtt
tggtggctcaaaaatgtaattacgcactccagtgcaaaagcaatcgaca
gcaaattacatatttgtaatggtgtgcaattgaactgcttttaaaattg
aatagcaattaaattgttgcttggcaaacacgaacacacacagcccgaa
aaaaaaccagacaaaccactacagacagactcagacacacaccgataga
aacgctgctgcgccgacgcagctgcgctgcgaacttctcttcctcttct
cataatttcattcacacaccagcttttgtttgtattgaaattagtcgcg
gcggcgtaattaaacaatgcggtatttcgaaactcgcgctgagaacgac
cgacaataacaccaacacagacaggagcgcgcccttctcgcacgcagca
caaatttattataacacgtaaacaacaacgccgcaacgccgaacgcaac
ttatttttccggcagcgacgcgtccgcatacgtccgctcacgttaagtt
ccgcagagagaagttgttgaaaacataaacagaatcacttgttgcactc
tttgagaaaactggggctattgcggaaaaaaccaactaaaaatattgca
ggttaggggtactacgctcgattggcgtacggccaccacttttgcgact
tcactgttaaccgctaccttcatagagacttttacccgataaatgttat
gtagtttgactttctctgttaatcacaagaaaaaatattgtggaaatta
aaattatctcaaactcaataaggaaataataatatatacacctatgttt
tatagaagtcaacagtaaataagttatttggaaaaccattgtagccgtt
taaataaatctccttgagtgtgttttaaataacggtcattaagtatatt
acttggccctctgaatttcttgaattacaccattttttgaaataaatca
atccaaaagactactttttggtggcaaatgaactgcataaaaagtaaca
aaagaaatatgtttttgaaataacagtatagctgaagtgtattaaaaaa
taccgtcatatgagcgacccgctgttaccgcttcgctgcgaatgacaaa
acgggctgagcaagaaaatggcgtagaaggcgacgaaaattcgtttcac
tcgtgaagaaaacctcgataactgaggaatacagctgggatttaaagag
catattcgaactacaagcagagatgtttcctggtggaaacggaaacgcc
gatttgggctacaacaagcatgcccacgtccatggacttggacaacatg
gccatgggcacaaccataatcacaatcagttcctgcgcagcccccacca
ccccccacacatttttcactgccctccgggggcggtcagggcatggtga
cgcccatggtagccgccggcctgccgctcgccatgcagggtggcgttgg
catcgattggcgcagctcgcccagcaatggatcc

The sequence for the VEGF-A basal promoter region:

tgtttagaagatgaaccgtaagcctaggctagaactgagggagcctact
actcccacccttccgagggttggcggcaggactgggcagctggcctacc
tacctttctgaatgctagggtaggtttgaatcaccatgccggcctggcc
cgcttctgcccccattggcaccctggcttcagttccctggcaacatctc
tgtgtgtgtgtgtgtgtgtgagagagagagatcaggaggaacaagggcc
tctgtctgcccagcagttgtctctccttcagggctctgccagactacac
agtgcatacgtgggtttccacaggtcgtctcactccccgccactgacta
actccagaactccacccccgttctcagtgccacaaatttggtgccaaat
tctctccagagaagcctctctggaaacttcccagaggatcccattcacc
ccagggccctagctcctgatgactgcagatcagacaagggctcagataa
gcatactcccccccccccgtaaccccctccccacatataaacctacagt
tatgcttccgaggtcaaacacgcaactttttgggtgtgtgtgtatgtca
gaaacacgcaattatttgggagctcaaagtctgccgcactcaagaatca
tctctcaccccctttccaagacccgtgccatttgagcaagagttggggt
gtgcataatgtagtcactagggggcgctcggccatcacggggagatcgt
aacttgggcgagccgagtctgcgtgagggaggacgcgtgtttcaatgtg
agtgcgtgcatgctgtgtgtgtgtgtgtagtgtgtgtgtgaggtggggg
agaaagccaggggtcactctagttgtccctatcctcatacgttcctgcc
agctctccgccttccaacccctactttctcctatatcctgggaaaggga
attgtcttagaccctgtccgcatataacctcactctcctgtctcccctg
attcccaatactctgggattcccagtgtgttcctgagcccatttgaagg
ggtgcacagataattttgaggccgtggaccctggtaaggggtttagctt
tccatttcgcggtagtggcctaggggctccccgaaaggcggtgcctggc
tccaccagaccgtccccggggcgggtctgggcggggcttgggggtggag
ctagatttcctctttttcttccaccgctgttaccggtgagaagcgcaga
ggcttggggcagccgagctgcagcgagcgcgcggcactgggggcgagct
gagcggcggcagcggagctctgtcgcgagacgcagcgacaaggcagact
atcagcggactcaccagcccgggagtctgtgctctgggatttgatattc
aaacctcttaatttttttttcttaaactgtattgttttacgctttaatt
tatttttgcttcctattcccctcttaaatcgtgccaacggtttgaggag
gttggttcttcactccctcaaatcacttcggattgtggaaatcagcaga
cgaaagaggtatcaagagctccagagagaagtcaaggaagagagagaga
gaccggtcagagagagcgcgctggcgagcgaacagagagagggacaggg
gcaaagttgacttgaccttgcttttgggggtgaccgccagagcgcggcg
tgacctcccccttcgatcttgcatcggaccagtcgcgctgacggacaga
cagacagacaccgcccccagccccagcgcccacctcctcgccggcggcc
tgccgacggtggacgcggcggcgagccgagaaaccgaagcccgcgcccg
gaggcgggtggagggggtcggggctcgcgggattgcacggaaacttttc
gtccaacttctgggctcttctcgctccgtagtagccgtggtctgcgccg
caggagacaaaccgatccggagctgggagaaggctagctcggccctgga
gaggccggggcccgagaagagaggggaggaaggaagaggagagggggcc
acagtgggcgctcggctctcaggagccgagctcatggacgggtgaggcg
gccgtgtgcgcagacagtgctccagccgcgcgcgcgccccaggccccgg
cccgggcctcggttccagaagggagaggagcccgccaaggcgcgcaaga
gagcgggctgcctcgcagtccggagccggagagagggagcgcgagccgc
cgcggccccggacggcctccgaaacc

Reporters

A reporter gene encodes an assayable product (e.g. chloramphenicol acetyl transferase (CAT)). A reporter is used to report activated gene expression by providing an easily detectable protein product (e.g., an enzymatic activity). The reporter gene of the present invention can have additional nucleic acids at both ends or at one end of the reporter gene sequence.

Examples of reporters that can be used in the present invention are CAT, lacZ, luciferase, Red Fluorescent Protein (RFP) and derivatives thereof, Green Fluorescent Protein (GFP) and derivatives thereof, Blue Fluorescent Protein and derivatives of, Cyan Fluorescent Protein and derivatives thereof, emerald GFP, mGFP5er, Yellow Fluorescent Protein and derivatives thereof, Propidium iodide, alkaline phosphatase, or any other detectable enzymatic activity, binding activity, or detectable RNA transcript. Additional examples of reporters are contained in the chart below.

In addition, indirect reporters can be used in the present invention. A secondary protein or compound can be used that interacts with the reporter protein and is labelled with a fluorchrome, radioactivity, or any of the known labelling substances known to one skilled in the art. The secondary protein could be a capture antibody that interacts with the reporter and is coupled to a label.

Detection Means

One way of detecting the enzymatic activity of a reporter protein is with the naked eye. One can see the green color, for example, of a green fluorescent protein that is expressed in a cell. Any of the proteins in which the result of their expression causes a color to appear can be seen by the naked eye, i.e. without the aid of a microscope or other device.

Another way of detecting the enzymatic activity of a reporter protein is using laser scanning microscopy. The key principle of laser scanning microscopy is that the sample is illuminated with a focused spot of laser light and the image is built up by scanning the spot over the field of view. This optical set up offers great flexibility in image acquisition strategies. In particular it enables production of optical section images, that is images in which light from out-of-focus regions does not contribute to the image. Optical section imaging has a wide range of applications in microscopy and allows the production of animated 3D projections. There are two distinctive methods of producing optical section LSM images—confocal microscopy and multi-photon excitation.

Multi-photon microscopy is an optical sectioning technique that uses infra-red light to excite fluorescent probes usually excited by UV or visible light. Excitation is restricted to a very tiny volume in the sample. Multi-photon excitation works by using short (femtosecond) pulses of low energy light to excite the standard fluorescent dyes. The photon density of the pulse is so high in the focal volume, that each fluorescent molecule now absorbs two photons whereas in conventional fluorescence imaging one photon is absorbed. The combined energies of two low energy photons is equivalent to the energy of one high energy photon. The fluorescent molecule does not know the difference and emits fluorescence in the same way as normal. The fall off of photon density outside the focal volume is so steep that nothing outside of it is excited. Thus, optical sectioning with multi-photon imaging is intrinsic to the excitation process. It follows that any fluorescence detected originates from that volume only allowing extremely efficient direct detection of all the signal (including scattered light) without any need for confocal apertures. Therefore, one way of detecting the enzymatic activity of a reporter protein is using a multi-photon fluorescence detection system, such as BioRad Radiance2100MP.

There are numerous sources of information regarding laser scanning microscopy. Examples are provided below: Fluorescence Microscopy of Living Cells in Culture, Part A, Editors Yu-Li Wang & D. Lansing-Taylor, Methods in Cell Biology Vol 29; Fluorescent and luminescent probes for biological activity. A practical guide to technology for quantitative real-time analysis, Editor W. T. Mason; Multidimensional Microscopy, Editors P. C. Cheng, T. H. Lin, W. L. Wu, J. L. Wu, Springer-Verlag 1994; Confocal Microscopy Methods and Protocols, Editor Stephen W. Paddock, Humana Press, 1999; and Handbook of Biological Confocal Microscopy (2nd Edition), Editor James B. Pawley, Plenum Press 1995.

Excitation and emission maxima for various of the fluorescent proteins and fluorochromes that are commonly used and can be used in the present invention are provided below:

| Fluorochrome | Excitation maximum | Emission maximum |
|---|---|---|
| | wavelength (nm) | wavelength (nm) |
| Blue fluorescent protein | 380 | 440 |
| Cyan fluorescent protein | 434 | 477 |
| Green fluorescent protein | 489 | 508 |
| emeraldGFP | 485-488 | 510 |
| mGFP5er | 405 and 477 | 510 |
| Yellow fluorescent protein | 514 | 527 |
| Red fluorescent protein | 558 | 583 |
| Propidium iodide | 540 | 610 |

Transcription Factor Families

Examples of transcription factor families that can be used in the present invention are: bHLH family, homeobox family, winged-helix family, helix-turn-helix (HTH) superfamily, Y-box family, T-box family, leucine zipper family, zinc-finger family, Paired box family, chromodomain family, and nuclear receptor family. These families can be present in or added to the cellular or acellular environment in which the regulatory assay is conducted.

Transcription Factors

Examples of transcription factors that can be used in the present invention are: Abd-B, Adf-1, bcd (bicoid ), Broad-Complex Z1, Broad-Complex Z2, Broad-Complex Z3, Borad-Complex Z4, CF1/USP (chorion factor 1), CF2-II, Croc, cut, Dfd (deformed), d1 (dorsal), E74A, Elf-1 (CP2), En (Engrailed), Evenskipped (Eve), Ftz (fushi tarazu), GCM (glial cells missing), Hairy, Hb (Hunchback), HSF (heat shock factor), Kr (Kruppel), Runt, Sn (Snail), STAT, Su(H) (Suppressor of Hairless), Su(Hw) Suppressor of Hairy wing), Ttk (tramtrack), Ubx (Ultrabithorax), AhR (aryl hydrocarbon dioxin receptor), AML-1a, AP-1, AP-2, AP-4, ARP-1 (apolipoprotein AI regulatory protein 1), Arnt, ATF (activating transcription factor), Bra (Brachyury), Barbie (barbiturate-inducible element), Brn-2, CdxA, C/EBP alpha, C/EBP beta, c-Ets-1, CHOP-C/EBP heterodimer, Clox, c-Myb, COMP1, COUP-TF —HNF4 heterodimer, CP2, CRE-BP1 -c-jun heterodimer, CRE-BP1, CREB, CBP (CCAAT binding protein), CDP, c-Rel, delta EF1, E2F, E2, E47, Egr-1/Krox-24/NGFI-A, Egr-2, Egr-3, Elk-1, ER (estrogen receptor), Evi-1, GATA-1, GATA-2, GATA-3, GC box binding factor, Gfi-1 (growth factor independence 1), Glucocorticoid receptor, HEN1, HFH-1, HFH-2, HLF (hepatic leukemia factor), HNF1 (hepatocyte nuclear factor 1), HNF3beta (hepatocyte nuclear factor 3 beta), HNF4 (hepatocyte nuclear factor 4), Hox factors (Hox-1.3 et cetera), HSF1 (heat shock factor), HSF2, Ik-1 (Ikaros), Ik-2, Ik-3, IRF-1 (interferon regulatory factor 1), IRF-2, Lyf-1, Max, MEF-2, c-Myb, Myc-Max heterodimer, MyoD, myogenin/NF-1, paraxis, scleraxis, Thing1, Thing2, HAND, MZF1, NF-1, NF-E2, NFkB (nuclear factor kappa B), NF-Y binding factor (Y-box binding factor), Nkx family (Nkx-2.5 et cetera), NRF-2 (nuclear respiratory factor 2), NRSF (neuron-restrictive silencing factor), Oct-1 (octamer factor 1), Olf-1 (olfactory neuron-specific factor), p300, p53 (TP53), TP73, Pax family (Pax-2, Pax-6, Pax-8 et cetera), BSAP (B-cell specific activating protein), Pbx-1, PPARalpha/RXR-alpha heterodimer, ROR alpha 1 (RAR-related orphan receptor alpha 1), RREB-1 (Ras-responsive element binding protein 1), RSRF4 (related to serum responsive factor C4), SEF-1, Sox family (Sox-5 et cetera), Sp1 (stimulating protein 1), SREBP-1 (sterol regulatory element-binding protein 1), SRF (serum responsive factor), SRY, Staf, STAT family (signal transducer and activator of transcription 1) (STAT-1, STAT-3 et cetera), thyroid hormone receptor, v-ErbA, Tal-1 alpha/E47 heterodimer, Tal-1 beta/E47 heterodimer, MyoD/E47 heterodimer, TBP (TATA binding protein), Tax/CREB complex, Thing1/E47 heterodimer, POU factor family (Tst1-Oct-6 e.g.), USF (upstream stimulating factor), VBP (vitellogenin promoter-binding protein), v-Jun, v-Maf, v-Myb, XBP-1 (X-box binding protein), and XFD-1 (Xenopus forkhead domain factor 1). These factors can be present in or added to the cellular or acellular environment in which the regulatory assay is conducted.

Vectors

Vectors that can be used in the present invention are described below. As used herein, the term “vector” refers to a nucleic acid molecule capable of transporting another nucleic acid to which it has been linked. One type of vector is an episome, i.e., a nucleic acid capable of extra-chromosomal replication. Other vectors are capable of autonomous replication and/expression of nucleic acids to which they are linked. Vectors capable of directing the expression of genes to which they are operably linked are referred to herein as “expression vectors.” In general, expression vectors of utility in recombinant DNA techniques are often in the form of “plasmids” which refer to circular double stranded DNA loops which, in their vector form are not bound to the chromosome. In the present specification, “plasmid” and “vector” are used interchangeably. In addition, the invention is intended to include other forms of vectors which serve equivalent functions and which become known in the art subsequently hereto.

Vectors can be used for the expression of polynucleotides and polypeptides. Generally, such vectors comprise cis-acting control regions effective for expression in a host operably linked to the polynucleotide to be expressed. Appropriate trans-acting factors either are supplied by the host, supplied by a complementing vector, or supplied by the vector itself upon introduction into the host.

In certain circumstances, the vectors provide for specific expression. Such specific expression may be inducible expression, expression only in certain types of cells, or both inducible and cell-specific. Vectors can be induced for expression by environmental factors that are easy to manipulate, such as temperature and nutrient additives. A variety of vectors such as constitutive and inducible expression vectors for use in prokaryotic and eukaryotic hosts, are well known and employed routinely by those of skill in the art.

A great variety of vectors can be used in the invention. Such vectors include chromosomal, episomal, virus-derived vectors, vectors derived from bacterial plasmids, from bacteriophage, from yeast episomes, from yeast chromosomal elements, from viruses such as baculoviruses, papovaviruses, such as SV40, vaccinia viruses, adenoviruses, fowl pox viruses, pseudo-rabies viruses and retroviruses, and vectors derived from combinations thereof, such as those derived from plasmid and bacteriophage genetic elements, such as cosmids and phagemids. Generally, any vector suitable to maintain, propagate or express polynucleotides in a host may be used.

The following vectors, which are commercially available, are provided by way of example. Among vectors for use in bacteria are pQE70, pQE60, and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Eukaryotic vectors available are pWLNEO, pSV2CAT, pOG44, pXT1, and pSG available from Stratagene; and pSVK3, pBPV, pMSG, and pSVL available from Pharmacia. These vectors are listed solely by way of illustration of the many commercially available and well known vectors that are available to those of skill in the art for use in accordance with the present invention. It will be appreciated that any other plasmid or vector suitable for, for example, introduction, maintenance, propagation, and/or expression of a polynucleotide or polypeptide of the invention in a host may be used in this aspect of the invention.

The appropriate DNA sequence may be inserted into the vector by any of a variety of well-known and routine techniques. In general, a DNA sequence for expression is joined to a vector by cleaving the DNA sequence and the vector with one or more restriction endonucleases and then joining the restriction fragments together using T4 DNA ligase. Procedures for restriction and ligation that can be used are well known and routine to those of skill in the art. Suitable procedures in this regard, and for constructing vectors using alternative techniques, which also are well known and routine to those skilled in the art, are set forth in great detail in Sambrook et al. cited elsewhere herein.

The sequence in the vector is operably linked to appropriate expression control sequence(s), including, for instance, a promoter to direct mRNA transcription.

It should be understood that the choice and/or design of the vector may depend on such factors as the choice of the host cell to be transformed and/or the type of protein(s) desired to be expressed. Moreover, the vector's copy number, the ability to control that copy number, and the expression of any other proteins encoded by the vector, such as antibiotic markers, should also be considered. Expression vectors can be used to transfect cells and thereby replicate regulatory sequences and produce proteins or peptides, including those encoded by nucleic acids as described herein.

Operably Linked

Operably linked is intended to mean that a first nucleotide sequence, for example a regulatory module, is linked to another sequence, for example an insulator, in a manner in which the first sequence and second sequence, and possibly more sequences, act together to obtain a desired effect.

Genetic Engineering of Cells

Host cells can be genetically engineered to incorporate polynucleotides and express polypeptides of the present invention. For instance, polynucleotides may be introduced into host cells using well known techniques of infection, transduction, transfection, transvection, and transformation. The polynucleotides may be introduced alone or with other polynucleotides. Such other polynucleotides may be introduced independently, co-introduced, or introduced joined to the polynucleotides of the invention.

Thus, for instance, polynucleotides of the invention may be transfected into host cells with another, separate, polynucleotide encoding a selectable marker, using standard techniques for co-transfection and selection in, for instance, mammalian cells. In this case the polynucleotides generally will be stably incorporated into the host cell genome.

In addition, the polynucleotides may be joined to a vector containing a selectable marker for propagation in a host. The vector construct may be introduced into host cells by the aforementioned techniques. Generally, a plasmid vector is introduced as DNA in a precipitate, such as a calcium phosphate precipitate, or in a complex with a charged lipid. Electroporation also may be used to introduce polynucleotides into a host. If the vector is a virus, it may be packaged in vitro or introduced into a packaging cell and the packaged virus may be transduced into cells. A wide variety of techniques suitable for making polynucleotides and for introducing polynucleotides into cells in accordance with this aspect of the invention are well known and routine to those of skill in the art. Such techniques are reviewed at length in Sambrook et al., which is illustrative of the many laboratory manuals that detail these techniques. In addition, the vector may be, for example, a plasmid vector, a single or double-stranded phage vector, a single or double-stranded RNA or DNA viral vector. Such vectors may be introduced into cells as polynucleotides, such as DNA, by well known techniques for introducing DNA and RNA into cells. The vectors, in the case of phage and viral vectors may be introduced into cells as packaged or encapsidated virus by well known techniques for infection and transduction. Viral vectors may be replication competent or replication defective. In the latter case viral propagation generally will occur only in complementing host cells.

As used herein, the term “transfection” means the introduction of a nucleic acid, e.g., an expression vector, into a recipient cell by nucleic acid-mediated gene transfer. “Transformation,” as used herein, refers to a process in which a cell's genotype is changed as a result of the cellular uptake of exogenous DNA or RNA. For example, a transformed cell expresses a recombinant form of a polypeptide or, where anti-sense expression occurs from the transferred gene, the expression of a naturally-occurring form of a protein is disrupted.

Transfection can be either transient transfection or stable transfection. Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis, et al., Basic Methods In Molecular Biology (1986).

Control and Test Pathways

Examples of the types of pathways that can be used in the present invention are: the MAP kinase/Ras pathways; Notch pathways; EGF pathways; TGF-beta superfamily pathways; cAMP pathways (for example TSH, ACTH LH, adrenaline, parathormone, adrenaline, glucagon, vasopressin); Tyrosine Kinase transmembrane receptor pathways; IP3 pathways; and Trimeric G protein coupled receptor pathways. This list is merely an exemplary list. These pathways are complex pathways that interact with other pathways in the cell. Articles are provided below that further describe various pathways that can be used in the present invention.

The signalling pathway for erythropoietin is described in the following articles: Cheung, J. Y., Miller, B. A., Molecular mechanisms of erythropoietin signaling, Nephron 87(3):215-22 (2001); Wilson, I. A. and Jolliffe, L. K., The structure, organization, activation and plasticity of the erythropoietin receptor, Curr Opin Struct Biol 9(6):696-704 (1999); Watowich, S. S., Activation of erythropoietin signaling by receptor dimerization, Int J Biochem Cell Biol 31(10):1075-88 (1999); Bunn, H. F., et al., Erythropoietin: a model system for studying oxygen-dependent gene regulation, J Exp Biol 201 (Pt 8):1197-201 (1998); and Damen, J. E. and Krystal, G., Early events in erythropoietin-induced signaling Exp Hematol 24(13):1455-9 (1996). The β-adrenergic receptor is described in Caron, M. G. and Lefkowitz, R. J., Catecholamine receptors: structure, function, and regulation, Recent Prog. Horn. Res. 48:277-290 (1993). The receptor tyrosine kinase signalling pathway is described in Fantl, W. J., et al., Signalling by receptor tyrosine kinases, Annu. Rev. Biochem. 62:453-481 (1993). The TGF-β family is described in Massague, J. L., et al., The TGF-β family and its composite receptors, Trends Cell. Biol. 4:172-178 (1994). Ras signalling is described in Nishida, E. and Gotoh, Y., The MAP kinase cascade is essential for diverse signal transduction pathways, Trends Biochem. Sci. 18:128-131 (1993). The insulin signalling cascade is described in Rosen, O. M., After insulin binds, Science 237:1452-1458 (1987). Hormone signalling in yeast is described in Levitzki, A., Transmembrane signaling to adenylate cyclase in mammalian cells and in Saccharomyces cerevisiae, Trends Biochem. Sci. 13:298-303 (1988). Other articles describing signal transduction include: Brindle, P., et al., Protein-kinase-A-dependent activator in transcription factor CREB reveals new role for CREM repressors, Nature 364:821-824 (1993); Darnell, J. E., et al., Jak-STAT pathways and transcriptional activation in response to IFNs and other extracellular signalling proteins, Science 264:1415-1420 (1994); and Hagiwara, M., et al., Transcriptional attenuation following cAMP induction requires PP-1 mediated dephosphorylation of CREB, Cell 70:105-113 (1992).

A generalized signal transduction pathway is shown in FIG. 5.

Libraries

One approach to finding lead drug molecules is to assemble and then screen large databases of chemical compounds. Significant collections already exist, and it is possible to purchase large numbers of commercially available compounds. Libraries can be obtained from commercially available sources such as Houghton Pharmaceuticals, Affymax, Chiron, Isis Pharmaceuticals, Gilead Sciences, Nexagen, Selectide, and Warner Lambert, among others. Types of libraries are, for example, peptide libraries, oligonucleotide libraries, carbohydrate libraries, and synthetic organic libraries.

In addition, PNA libraries can be used in the present invention. PNAs have been shown to be useful in antisensense and hybridization technology. The applications of PNAs are further described in the following articles: Ørum, H., et al., Peptide Nucleic Acid, pp. 29-48, Biotechniques Books, Div. Eaton Publishing, Birkhäuser Boston, 1997; Nielsen, P. E., Applications of peptide nucleic acids, Current Opinion in Biotechnology 10:71-75 (1999); and Nielsen, P. E., Peptide nucleic acid (PNA). From DNA recognition to antisense and DNA structure, Biophysical Chemistry 68:103-108 (1997).

An example of a widely used database of organic compounds is the Cambridge database of X-ray crystallographic data, which contains nearly 100,000 organic compound structures. Other databases are available of 2D chemical structures, and reliable tools for generating 3D structures from 2D database-derived connection tables allow these to routinely be converted into 3D for drug design studies.

Computational screening of these or other compound databases allows one to take a lead pharmacophore or compound and probe the database for other molecules that are structurally similar to the query.

The ability to “mine” chemical structure databases is revolutionizing the search for new drugs. The cycle of search, synthesis, screen, and analysis of potentially thousands of new compounds can be reduced from years to months.

Tissue Culture

The engineered host cells can be cultured in conventional nutrient media, which may be modified as appropriate for, inter alia, activating promoters, selecting transformants, or amplifying genes, for example. Culture conditions, such as temperature, pH, and the like, can be chosen and altered if needed making the conditions suitable for replication and/or expression of polynucleotides of the present invention, as will be apparent to those of skill in the art.

Acellular Systems

An example of an acellular system capable of being used in the present invention is a cell free extract. For example, a cell free extract can be obtained by rupturing the cells and removing all particulate matter.

Cellular Systems

Examples of human and animal cell lines that can be used in the present invention are: 10T1/2, 1G1, 22RV1, 23132/87, 293, 2A1, 2E10-H2, 2HX-2, 2M6, 32D, 380, 3T3, 3T6, 42-MG-BA, 4H1-A7, 5637, 639-V, 647-V, 697, 7-TD-1, 72A1, 8-MG-BA, 8305C, 8505C, A-10, A-2, A-204, A-427, A-431, A-498, A-549, A-S-30D, A4-1025, A4-1077, A4-840, A4-951, AC-1M32, AC-1M46, AC-1M59, AC-1M81, AC-1M88, ACH1P, AM-C6SC8, AN3-CA, B-16V, B-CPAP, B9, B95-8, BA-D5, BA-F8, BA/F3, BAG-12G2, BAG-85D10, BC-3C, BC3H1, BD-215, BE-13, BEN, BETA-TC-3, BEWO, BF-32, BF-34, BF-45, BF-F3, BF-G6, BFTC-905, BFTC-909, BHK-21, BHT-101, BHY, BL-41, BL-70, BM-1604, BONNA-12, BPH-1, BT-474, BT-B, BTI-EAA, BV-173, C-433, C6-BU-1, C7, CA-46, CACO-2, CADO-ES1, CAKI-1, CAKI-2, CAL-120, CAL-12T, CAL-148, CAL-27, CAL-33, CAL-39, CAL-51, CAL-54, CAL-62, CAL-72, CAL-78, CAL-85-1, CAPAN-1, CAPAN-2, CAT-13.0B10, CAT-13.1E10, CAT-13.6E12, CAT-13.9C1, CCRF-CEM, CF-10H5, CF-1D12, CGTH-W-1, CHO-DHFR[−], CHO-K1, CHP-126, CMK, CML-T1, COLO-206F, COLO-320, COLO-677, COLO-678, COLO-679, COLO-680N, COLO-699, COLO-704, COLO-720L, COLO-783, COLO-800, COLO-818, COLO-824, COLO-849, COS-1, COS-7, CPC-N, CRO-AP2, CRO-AP3, CRO-AP5, CTV-1, CX-1D-11, D-36, D10.G4.1, D3, DA-1, DAN-G, DAUDI, DBTRG-05MG, DEL, DG-75, DK-MG, DLD-1, DMBM-2, DOHH-2, DU-145, DU-4475, DV-90, EB-1, EBL, ECV-304, EFE-184, EFM-19, EFM-192A, EFM-192B, EFM-192C, EFO-21, EFO-27, EGI-1, EHEB, ELM-I-1, EM-2, EM-3, EOL-1, EPLC-272H, ESS-1, EVSA-T, F1-652, F4/4.K6, F9FDCP-1, FDCP-Mixcl.A4, FLK-BLV-044, FU-OV-1, G/G, GAMG, GDM-1, GH3, GH4-C1, GIRARDIHEARTC2, GIRARDIHEARTC7, GM-7373, GMS-10, GOS-3, GRANTA-519, H25B10, HAP-T1, HC-1, HCC-366, HCT-15, HD-MY-Z, HDLM-2, HDQ-P1, HEL, HELA, HELA-S3, HEP-3B, HEP-G2, HEPA1-6, HH-16cl.2/1, HH-16.cl.4, HKT-1097, HL-60, HN, HPB-ALL, HPD-1NR, HPD-2NR, HSB-2, HT-1080, HT-1376, HT-29, HUP-T3, HUP-T4, IEC-6, IGR-1, IGR-37, IGR-39, IM-9, IMR-32, IPC-298, IPL-LD-65Y, J-774A.1, JAR, JEG-3, JK-1, JOSK-I, JOSK-M, JTC-15, JTC-27, JURKAT, JVM-13, JVM-2, JVM-3K-562, KARPAS-299, KARPAS-422, KARPAS-45, KASUMI-1, KB, KB-3-1, KB-V1, KE-37, KELLY, KG-1, KG-1a, KM-H2, KMOE-2, KPL-1, KU-19-19, KU-812, KYSE-140, KYSE-150, KYSE-180, KYSE-270, KYSE-30, KYSE-410, KYSE-450, KYSE-510, KYSE-520,KYSE-70, L-1210, L-363, L-428, L-5178-Y, L-540, L-929, L138.8A, LAMA-84, LAMA-87, LAT, LCL-HO, LCL-WEI, LCLC-103H, LCLC-97TM1, LF-CL2A, LN-405, LNCAP, LOU-NH91, LOUCY, LOVO, LP-1, LXF-289, M-07e, M1,M3E3/C3, MB-020, MB-021, MB-03, MB-04, MB-L11, MB-L2MC-116, MC3T3-E1, MCF-7, MDA-MB-453, MDBK, MEG-01, MEL-HO, MEL-JUSO, MFE-280, MFE-296, MFE-319, MFM-223, MH-7777A, MH1C1, MHEC5-T, MHH-CALL-2, MHH-CALL-3, MHH-CALL-4, MHH-ES-1, MHH-NB-11, MHH-PREB-1, MKN-45, ML-2, MMQ, MN-60, MOLT-13, MOLT-14, MOLT-16, MOLT-17, MOLT-3, MOLT-4, MONO-MAC-1, MONO-MAC-6, MS-5, MSTO-211H, MT-3, MUTZ-1, MUTZ-2, MUTZ-3, MUTZ-5, MV4-11, N18TG2, N2-261, N3-36, N4TG3, NALM-1, NALM-6 NAMALWA, NAMALWA.CSN/70, NAMALWA.IPN/45, NAMALWA.KN2, NAMALWA.PNT, NB-4, NC-NC, NCI-H929, NEURO-2A, NIH-3T3, NK-92, NRK-49F, NRK-52E, NS20Y, NUC-1, NUC-5, OCI-AML2, OCI-AML5, OMEGA-E, OPM-2, OTH-74D4, P-19, P-388D1(IL-1), P-815, P12-ICHIKAWA, P3/NSI/1-AG4-1, PA-TU-8902, PA-TU-8988S, PA-TU-8988T, PAB-100, PAB-122, PAB-1620, PB-1, PC-12, PC-3, PEER, PF-382, PLB-985, PR-1, PSI-2, PYSR1, RAJI, RBL-1, RBL-2H3, RD-ES, RED-1, RED-4, RED-5, RED-6, REH, RGE, RH-30, RLC-18, RLD-1, RMB-1, RPMI-2650, RPMI-7951, RPMI-8226, RPMI-8402, RSRT-112, RT-4, RTG-2, RV-C2, RVH-421, S-117SAO, S-2SBC-2, SBC-7, SC-71, SC-75, SCHNEIDER-2, SCLC-21H, SCLC-22H, SD-1, SER-W3, SF-158, SF-21, SF-9, SGE-1, SH-SY5Y, SIG-M5, SIMA, SISO, SK-HEP-1, SK-MEL-1, SK-MEL-3, SK-MEL-30, SK-MES-1, SK-MM-2, SK-N-MC, SKW-3, SNB-19, SOM-4D10, SP2/0-AG14, SPC-BM-36, SPI-801, SPI-802, SR-4987, SR-786, ST-2, SU-DHL-1, SU-DHL-4, SUP-B15, SUP-T1, SW-1710, SW-403, SW-480, SW-948, T-24, TANOUE, TCC-SUP, TE-671, TF-1, TFK-1, THB-5, THP-1, TI-1, TI-4, TMM, TN-368, U-138-MG, U-2197, U-266, U-698-M, U-937, UT-7, V-79, VA-ES-BJ, VERO-B4, VIP-VIIIC8, VLMVM-CUB1, WEHI-164S, WEHI-3B, WERI-RB-1, WMP-2, WSU-NHL, X63AG8.653, XC, XTH-2, Y-79, YAC-1, YAPC, and YT.

Primary cells, secondary cells, and cell strains can be used in the present invention. As used herein, the term primary cell includes cells present in a suspension of cells isolated from a vertebrate tissue source (prior to their being plated, i.e., attached to a tissue culture substrate such as a dish or flask), cells present in an explant derived from tissue, both of the previous types of cells plated for the first time, and cell suspensions derived from these plated cells. The term secondary cell or cell strain refers to cells at all subsequent steps in culturing. That is, the first time a plated primary cell is removed from the culture substrate and replated (passaged), it is referred to herein as a secondary cell, as are all cells in subsequent passages. Secondary cells are cell strains which consist of secondary cells which have been passaged one or more times. A cell strain consists of secondary cells that: 1) have been passaged one or more times; 2) exhibit a finite number of mean population doublings in culture; 3) exhibit the properties of contact-inhibited, anchorage dependent growth (anchorage-dependence does not apply to cells that are propagated in suspension culture); and 4) are not immortalized.

Primary and secondary cells to be used in the present method can be obtained from a variety of tissues and include all cell types which can be maintained in culture. For example, primary and secondary cells which can be transfected by the present method include fibroblasts, keratinocytes, epithelial cells (e.g., mammary epithelial cells, intestinal epithelial cells), endothelial cells, glial cells, neural cells, formed elements of the blood (e.g., lymphocytes, bone marrow cells), muscle cells and precursors of these somatic cell types. Primary cells can be obtained from the individual to whom the transfected primary or secondary cells are administered. However, primary cells can be obtained from a donor (other than the recipient) of the same species or another species (e.g., mouse, rat, rabbit, cat, dog, pig, cow, bird, sheep, goat, horse).

Immortalized cells can also be transfected by the present method and used for either protein production or gene therapy. Examples of immortalized human cell lines useful for protein production or gene therapy include, but are not limited to, HT1080, HeLa, MCF-7 breast cancer cells, K-562 leukemia cells, KB carcinoma cells and 2780AD ovarian carcinoma cells. Immortalized cells from other species (e.g., Chinese hamster ovary (CHO) cells or mouse L cells) can be used for in vitro protein production or gene therapy. In addition, primary or secondary human cells, as well as primary or secondary cells from other species which display the properties of gene amplification in vitro can be used for in vitro protein production or gene therapy.

Plant Systems

Vectors useful for genetic engineering in agriculture are described in Molecular Biology of Plant Tumors, Editors G. Kahl and J. S. Schell, Academic Press, Inc. New York, N.Y., 1982, pp. 1-597. Specifically, the Cauliflower Mosaic Virus and its use in plant genetic engineering, the plasmids of Rhizobium and symbiotic nitrogen fixation, and the transfer of symbiotic genes in Rhizobium are discussed.

Exogenous DNA

Exogenous DNA may be DNA that is normally expressed in the manipulated cell or DNA that is not normally expressed in the manipulated cell.

Exogenous DNA incorporated into primary, secondary or immortalized cells by the present method is: 1) DNA which encodes a translation or transcription product whose expression in cells is desired, or a portion of a translation or transcription product, such as a protein product or RNA product useful to treat an existing condition or prevent it from occurring; or 2) DNA which does not encode a gene product but is itself useful, such as a transcriptional regulatory sequence or DNA useful to treat an existing condition or prevent it from occurring.

DNA transfected into primary, secondary or immortalized cells can encode an entire desired product, or can encode, for example, the active or functional portion(s) of the product. The product can be, for example, a hormone, a cytokine, an antigen, an antibody, an enzyme, a clotting factor, a transport protein, a receptor, a regulatory protein, a structural protein, a transcription factor, an anti-sense RNA, or a ribozyme. Additionally, the product can be a protein or a nucleic acid which does not occur in nature (i.e., a novel protein or novel nucleic acid). The DNA can be obtained from a source in which it occurs in nature or can be produced, using genetic engineering techniques or synthetic processes. The DNA can encode one or more products. After transfection, the exogenous DNA is either transiently expressed or stably incorporated into the recipient cell's genome, from which it is expressed or otherwise functions. Alternatively, the exogenous DNA can be used to target DNA that exists episomally within cells.

Assays

There are a number of reporter assays, that can be used in the present invention. Assays can be performed in numerous different formats, such as a petri dish, a six-well dish, or a microtiter plate format. Examples of several assays that can be used in the present invention are provided below. Reporter assays enable rapid quantitative evaluation of physiological events.

The LacZ Reporter Assay can be used in the present invention. The E. coli lacZ gene encoding β-galactosidase is the classical histochemical reporter gene. β-galactosidase catalyzes the hydrolysis of X-Gal (5-bromo 4-chloro-3-indoyl-β-D-galactopyranoside) producing a blue precipitate that can be easily visualized under a microscope. The steps of a typical LacZ Reporter Assay assay are provided. 1. Remove media from plate. 2. Wash cells twice with PBS. 3. Dilute 5× Reporter Lysis Buffer to 1×. 4. Add Reporter Lysis Buffer and incubate for 15 minutes. 5. Scrape cells. 6. Remove cells to a clean tube, vortex and centrifuge for 2 minutes at 4° C. 7. Add 2× Assay Buffer and incubate at 37° C. for 30 minutes. 8. Stop the reactions. 9. Measure absorbance at 405 nm. This protocol can be modified as needed.

The PLAP Reporter Assay can also be used in the present invention. Plap is a human gene encoding placental alkaline phosphatase used as a histochemical reporter fairly recently. PLAP is a glycan phosphatidylinositol (GPI)-anchored protein which, unlike endogenous alkaline phosphatases, is very heat stable. PLAP catalyzes the hydrolysis of BCIP (5-bromo-4-chloro-3-indoyl-phosphate) producing a purple precipitate that can be easily visualized under a microscope.

The Luciferase assay can also be used in the present invention. Firefly luciferase is a widely used bioluminescent reporter because its enzyme activity is closely coupled to protein synthesis, and the luminescence assay is rapid, convenient and sensitive. Although various assay formulations for firefly luciferase have been described, the most widely used contains coenzyme A in addition to beetle luciferin and ATP. In a 1-10 second measurement, this assay provides linearity over a 100 million-fold concentration range with sensitivity greater than 10⁻²⁰ moles of enzyme. Recently, Renilla luciferase has also become widely used as a genetic reporter, although primarily as a co-reporter to firefly luciferase. Assay of Renilla luciferase is also rapid and linear, but the sensitivity is limited somewhat by autoluminescence. An assay format called the Dual-Luciferase® Reporter (DLR®) Assay has been designed to sequentially quantitate both firefly and Renilla luciferases from a single sample. The integration of the two luciferase assays provides an efficient means for incorporating an internal control into reporter measurements, or for analyzing two separate events in the same system. Bacterial luciferase, although the first luciferase to be used as a reporter, is generally used to provide autonomous luminescence in bacterial systems through expression of the lux operon. Ordinarily it is not useful for analysis in eukaryotic systems. U.S. Pat. Nos. 5,283,179, 5,641,641, and 5,650,289 describe a firefly luciferase assay method, which affords greater light output with improved kinetics as compared to the conventional assay.

With bioluminescence, the reporter measurements are nearly instantaneous, they are sensitive and quantitative, and typically there is no endogenous activity in the host cells to interfere with quantitation.

Luciferase genes have been cloned from bacteria, beetles (including firefly), Renilla, Aequorea, Vargula and Gonyaulax (a dinoflagellate). The luciferases from bacteria, firefly and Renilla have found general use as indicators of gene expression.

Bacterial luciferase is a dimeric enzyme of 80 kDa found in several marine bacteria and one species of terrestrial bacteria (Meighen, E. A., FASEB J. 7:1016 (1993), and Dunlap, P. V., Photochem. Photobiol. 54:1157 (1991)). The luminescence is generated from an oxidation reaction involving FMNH₂ and an aliphatic aldehyde to yield FMN, carboxylate and blue light of 490 nm.

Firefly luciferase is a commonly used bioluminescent reporters (de Wet, J. R., et al., PNAS USA 82:7870 (1985), Ow, D., et al., Science 234:856 (1986), and de Wet, J. R., et al., Mol. Cell. Biol. 7:725 (1987)). This monomeric enzyme of 61 kDa catalyzes a two-step oxidation reaction to yield light, usually in the green to yellow region, typically 550-570 nm. Firefly luciferase exhibits a close association between protein synthesis and enzyme activity, and the assay is rapid, sensitive and convenient.

Renilla luciferase is a 31 kDa monomeric enzyme that catalyzes the oxidation of coelenterazine to yield coelenteramide and blue light of 480 nm (Lorenz, W. W., et al., PNAS USA 88:4438 (1991), and Lorenz, W. W., et al., Bioluminescence and Chemiluminescence: Status Report, Editors John Wiley and Sons, Chicester, 191 (1993)). Renilla luciferase is further described in U.S. Pat. No. 5,292,658.

Although luminescence measurements in most research applications are 5-10 seconds per sample, in some cases assays of less than one second are performed allowing for thousands of assays per hour. Because of these general performance features and its applicability to virtually any host system, luciferase is a commonly chosen reporter.

Transgenic Animals

The present invention relates to transgenic animals having cells that contain portions of or a complete construct as shown in FIGS. 1 through 4 and described herein. Such transgenic animals represent for example, a model system for the study of EPO related disorders and/or the study of EPO based therapeutics.

A portion of a construct containing an endogenous EPO enhancer operably linked to a GFP coding sequence that is operably linked to an insulator can be integrated into the genome of a mouse through various techniques such as homologous recombination or random integration. The inserted portion will be placed in the same region of the mouse genome as the endogenous enhancer. The result of this insertion will be the ability to detect compounds that effect transcription and subsequent translation of the GFP gene by interacting with one or more of the players in the pathway that leads to activation of the EPO enhancer. In addition, the compound can directly effect the EPO enhancer. Another construct that can be used in the present invention is an EPO enhancer that has been made defective by a site direct mutation for example, operably linked to a GFP coding sequence, that is operably linked to an insulator. This construct, when properly integrated into the genome of a mouse could be used to study the effects of a defective enhancer on the expression of a gene in an in vivo system.

The term “animal” here denotes all mammalian species except human. It also includes an individual animal in all stages of development, including embryonic and fetal stages. Farm animals (pigs, goats, sheep, cows, horses, rabbits and the like), rodents (such as mice) and domestic pets (for example, cats and dogs) are included within the scope of the present invention.

A “transgenic” animal is any animal containing cells that bear genetic information received, directly or indirectly, by deliberate genetic manipulation at the subcellular level, such as by microinjection or infection with recombinant virus. “Transgenic” could encompass classical crossbreeding or in vitro fertilization, in addition to animals in which one or more cells receive a recombinant DNA molecule. This recombinant DNA molecule may be integrated within the animal's chromosomes. In addition, the present invention also contemplates the use of extrachromasomally replicating DNA sequences, such as might be engineered into yeast artificial chromosomes.

The term “transgenic animal” also includes a “germ cell line” transgenic animal. A germ cell line transgenic animal is a transgenic animal in which the genetic information has been taken up and incorporated into a germ line cell, therefore conferring the ability to transfer the information to offspring. If such offspring in fact possess some or all of that information, then they, too, are transgenic animals.

The transgenic animals of the present invention can be produced by introducing into single cell embryos DNA encoding for example the EPO enhancer—GFP —insulator construct described above, in a manner such that the polynucleotides are stably integrated into the DNA of germ line of cells of the mature animal and inherited in normal mendelian fashion. Advances in technologies for embryo micromanipulation now permit introduction of heterologous DNA into fertilized mammalian ova. For instance, totipotent or pluripotent stem cells can be transformed by microinjection, calcium phosphate mediated precipitation, liposome fusion, retroviral infection or other means, the transformed cells are then introduced into the embryo, and the embryo then develops into a transgenic animal. In a alternative method, developing embryos are infected with a retrovirus containing the desired DNA, and transgenic animals produced from the infected embryo.

In yet another method, the appropriate DNAs are coinjected into the pronucleus or cytoplasm of embryos, preferably at the single cell stage, and the embryos allowed to develop into mature transgenic animals. These techniques are well known. For instance, reviews of standard laboratory procedures for microinjection of heterologous DNAs into mammalian (mouse, pig, rabbit, sheep, goat, cow) fertilized ova include Hogan et al. Manipulating the Mouse Embryo (Cold Spring Harbor Press 1986); Krimpenfort et al., 1991, Bio/Technology 9:86; Palmiter et al., 1985, Cell 41:343; Kraemer et al., Genetic Manipulation of the Early Mammalian Embryo (Cold Spring Harbor Laboratory Press 1985); Hammer et al., 1985, Nature, 315:680; Purcel et al., 1986, Science, 244: 1281; Wagner et al., U.S. Pat. No. 5,175,385; and Krimpenfort et al., U.S Pat. No. 5,175,384.

The cDNA that encodes the above-described construct, for example, can be fused in proper reading frame under the transcriptional and translational control of a vector to produce a genetic construct that is then amplified, for example, by preparation in a bacterial vector, according to conventional methods. See, for example, the standard work: Sambrook, et al., Molecular Cloning: a Laboratory Manual (Cold Spring Harbor Press 1989). The amplified construct is thereafter excised from the vector and purified for use in producing transgenic animals.

The term “transgenic” as used herein additionally includes any organism whose genome has been altered by in vitro manipulation of the early embryo or fertilized egg or by any transgenic technology to induce a specific gene knockout. The term “gene knockout as used herein, refers to the targeted disruption of a gene in vivo with complete loss of function that has been achieved by any transgenic technology familiar to those in the art. Transgenic animals having gene knockouts can be those in which the target gene has been rendered nonfunctional by an insertion targeted to the gene to be rendered non-functional by homologous recombination. As used herein, the term “transgenic” includes any transgenic technology familiar to those in the art which can produce an organism carrying an introduced transgene or one in which an endogenous gene has been rendered non-functional or knocked out.

A transgene is any exogenous piece of DNA that is capable of being integrated into a genomic locus. The exogenous DNA can be derived from any species, or source. The DNA can be normally present in the genome of the animal or it can not be normally present in the genome of the animal. The transgene to be used in the practice of the subject invention is a nucleic acid sequence comprising any of the constructs described herein or portions thereof. Where appropriate, DNA sequences that encode proteins for example GFP but differ in nucleic acid sequence due to the degeneracy of the genetic code may also be used herein, as may truncated forms, allelic variants, and interspecies homologues.

Therapeutic Uses

The present invention can be used to identify new compounds useful in the treatment of various diseases. For example, compounds useful in the treatment of hypoxia related disorders can be identified using the methods of the present invention.

Hypoxia is a condition in which there is a reduction in the oxygen supply to tissues below physiological levels. Hypoxia plays a fundamental role in the pathophysiology of ischemic heart disease, cancer, stroke, chronic lung disease, congestive heart failure, and anemia. Revascularization therapeutics in the case of heart disease, angiogenic inhibitors in the case of cancer and tumor growth, and erythropoiesis inductors in the case of anemia are some examples of such possible compounds that can be identified by the methods of the present invention.

Hypoxia is an emerging area in the field of oncogenesis and holds great promise for developing new therapeutics aimed at disrupting vascularization (angiogenesis) of growing tumors. Under conditions of low-oxygen tensions, certain genes are induced by regulatory sequences acting as hypoxia sensors. These genes sometimes encode endothelial growth factors, which cause blood vessel growth into the region of low oxygen concentrations. Although these genes are often used during development and in tissue-repair they are often exploited in oncogenesis. Tumors are usually size-limited by insufficient blood supply unless they acquire the ability to induce angiogenesis. The entire physiological pathway and set of genes involved in angiogenesis is currently unknown. The methods of the present invention can be used to identify the genes involved in the pathway, and to identify compounds that can disrupt the progression of angiogenesis.

A compound useful in the treatment of a patient with a hypoxia related disorder could be identified using the methods of the present invention. For example, a method for identifying at least one compound that interacts with a test pathway comprising: providing a cell comprising an isolated nucleic acid comprising the coding region for a first reporter gene operably linked to a first control module, said first first control module being operably linked to a first regulatory module, said first regulatory module being operably linked to an insulator sequence, said insulator sequence being operably linked to a second regulatory module different than said first regulatory module, said second regulatory module being operably linked to a second control module, said second control module being operably linked to the coding region for a second reporter gene different than said first reporter gene, contacting the cell with at least one compound; monitoring the differences in the expression levels of the reporter genes, wherein said first reporter gene is operably linked to a control pathway and said second reporter gene is operably linked to a test pathway; whereby a difference in the expression levels of the reporter genes identifies a compound that interacts with the test pathway, and using said compound to treat a patient with a hypoxia related disorder.

Kits

The invention further relates to kits comprising one or more containers filled with one or more of the ingredients of the above-mentioned compositions of the invention. For example, cell lines containing a nucleic acid construct as shown in any of the figures and defined in the specification, can be shipped ready to use. In addition, the nucleic acid constructs themselves can be placed in a container and shipped ready to use. Associated with such container(s) can be instructions on how to use the kit(s).

The present invention is further described by the following examples. The examples are provided solely to illustrate the invention by reference to specific embodiments. These exemplifications, while illustrating certain specific aspects of the invention, do not portray the limitations or circumscribe the scope of the disclosed invention.

All examples were carried out using standard techniques, which are well known and routine to those of skill in the art, except where otherwise described in detail. Routine molecular biology techniques of the following examples can be carried out as described in standard laboratory manuals, such as Sambrook, et al., Molecular Cloning: A Laboratory Manual, 2nd Ed.; Cold Spring Harbor Laboratory Press, Cold Spring Harbor, N.Y. (1989), herein referred to as “Sambrook.”

### EXAMPLES

### Example 1

### Regulatory Assay Constructs

The following are examples of regulatory constructs that can be made and used in the present invention:

1. RFP<=(γ-globin promoter)—(EPO 3′ hypoxia enhancer)—(cHS4)—(VEGF hypoxia enhancer)—(γ-globin promoter)=>GFP

2. RFP<=(cytoplasmic actin promoter)—(cHS4)—(VEGF hypoxia enhancer)—(γ-globin promoter)=>GFP

3. RFP<=(γ-globin promoter)—(EPO 3′ hypoxia enhancer)—(cHS4)—(cytoplasmic actin promoter)=>GFP

4. RFP<=(γ-globin promoter)—(LBP-32 enhancer)—(cHS4) - (HMOX1 enhancer)—(γ-globin promoter)=>GFP

5. RFP<=(γ-globin promoter)—(relA hypoxia enhancer)—(cHS4)—(HMOX1 enhancer)—(γ-globin promoter)=>GFP

6. RFP<=(γ-globin promoter)—(PROC hypoxia enhancer)—(cHS4)—(HMOX1 enhancer)—(γ-globin promoter)=>GFP

7. RFP<=(γ-globin promoter)—(DELTEX hypoxia enhancer)—(cHS4)—(HMOX1 enhancer)—(γ-globin promoter)=>GFP

8. RFP<=(γ-globin promoter)—(COL4A1)—(cHS4)—(HMOX1 enhancer)—(γ-globin promoter)=>GFP

9. RFP<=(γ-globin promoter)—(GRAP)—(cHS4)—(HMOX1 enhancer)—(γ-globin promoter)=>GFP

10. RFP<=(γ-globin promoter)—(BTEγ-4 hypoxia enhancer)—(cHS4)—(HMOX1 enhancer)—(γ-globin promoter)=>GFP

11. RFP<=(γ-globin promoter)—(VEGF hypoxia enhancer)—(cHS4)—(HMOX1 enhancer)—(γ-globin promoter)=>GFP

12. RFP<=(γ-globin promoter)—(EPO 3′ hypoxia enhancer)—(cHS4)—(HMOX1 enhancer)—(γ-globin promoter)=>GFP

13. RFP<=(γ-globin promoter)—(EPO 3′ hypoxia enhancer)—(cHS4)—(VEGF hypoxia enhancer)—(γ-globin promoter)=>GFP

14. RFP<=(CCRdelta5 lymphocyte promoter)—(cHS4)—(cytoplasmic actin promoter)=>GFP

RFP and GFP are abbreviations for Red Fluorescent Protein and Green Fluorescent Protein, two reporters. cHS4 is the insulator sequence in these examples. The reporter genes and insulator sequences may be substituted without changing the spirit of the desired screen. The test and control reporter systems may lie either to the left or right of the insulator sequence in these examples.

The GFP to RFP signal ratio communicates when a compound specifically and functionally interacts with any component of either pathway. In these examples, the assays can be used to find small molecules that specifically upregulate, downregulate, or alter transcription of the EPO gene, for example, compared to the VEGF gene, or compared to some general housekeeping or common structural gene (e.g. cytoplasmic actin). The assay may also be used to find compounds that specifically downregulate, upregulate, or alter transcription of VEGF compared to control reporter genes. Each of these examples may be useful in screening for potential lead therapeutic compounds with applications in anemia, heart disease, or tumor progression, among others.

LBP-32 is an abbreviation for laminin binding protein, HMOX1 is an abbreviation for human monoxygenase 1, and COL4A1 is an abbreviation for collagen type-4 A1.

### Example 2

### Reporter Plasmid Construction

The following primers were used to amplify the corresponding regulatory constructs described above. The template for each of the primer pairs is human genomic DNA. Each primer pair amplifies the enhancer containing sequences used in the regulatory assay construct.

| | |
|---|---|
| Primer No.1: | |
| TTCAAGGACCCACTTACTCTGG | (LBP-32 top-1), |
| Primer No.2: | |
| TGTGAAGCTCTGCCAAGTACC | (LBP-32 top-2), |
| Primer No.3: | |
| TCCCACCTCATCTCCATAAGC | (LBP-32 bottom-1), |
| Primer No.4: | |
| GACTCCTAGAACATTGACACCC | (LBP-32 bottom-2), |
| Primer No.5: | |
| ACCCTTTAGAGCTTAGAGAGTCG | (HMOX1 top-1), |
| Primer No.6: | |
| TTAGAGAGTCGAAGAGGCAGG | (HMOX1 top-2), |
| Primer No.7: | |
| AGATAGAGGTTCCCTAAAACAGTGG | (HMOX1 bottom-1), |
| Primer No.8: | |
| TTCCCTAAAACAGTGGTGCGG | (HMOX1 bottom-2), |
| Primer No.9: | |
| AGGACCTGCTCCCTAGAACC | (re1A top-1), |
| Primer No.10: | |
| TGACTCAGTTTCCCCTCTGG | (re1A bottom-1), |
| Primer No.11: | |
| CAGAACAGATAGTGTAAAGAGTGC | (PROC top-1), |
| Primer No.12: | |
| CATTCCTGTATAGGGAGAAATATGG | (PROC top-2), |
| Primer No.13: | |
| GGTGAAGGTGGTTGGAGATCG | (PROC bottom-1), |
| Primer No.14: | |
| AGTGTGAAGAGGAGGACGAGC | (PROC bottom-2), |
| Primer No.15: | |
| CACCTACATACAGAGACTTGTGC | (DTX1 top-1), |
| Primer No.16: | |
| ACCTGTGTGCATGTGTGATTGTGC | (DTX1 bottom-1), |
| Primer No.17: | |
| ATTGTGCCTCTGCATGTGTGC | (DTX1 bottom-2), |
| Primer No.18: | |
| GTACATTTGGTGCGGAACTTGC | (COL4A1 top-1), |
| Primer No.19: | |
| ACAGTCACAAATTCCCAGAAACAGG | (COL4A1 bottom-1), |
| Primer No.20: | |
| GGATGAGTTTGCTTTAGGCTGG | (COL4A1 bottom-2), |
| Primer No.21: | |
| ACTGGGATGGGAAGAAAGTAAGG | (GRAP top-1), |
| Primer No.22: | |
| AAAGTAAGGGATCGGAACAGCG | (GRAP top-1), |
| Primer No.23: | |
| GGCTTAGGCCTCTGATATTTTCG | (GRAP bottom-1), |
| Primer No.24: | |
| TGATATTTTCGGAATTCGGGCACC | (GRAP bottom-1), |
| Primer No.25: | |
| AGCAGGAAGCATTCAGAGAGC | (EPO top-1), |
| Primer No.26: | |
| TCATTGACAAGAACTGAAACCACC | (EPO top-2), |
| Primer No.27: | |
| CCTGGGCAACATAGCAAGACC | (EPO bottom-1), |
| Primer No.28: | |
| CCTTGATGACAATCTCAGCGC | (EPO bottom-2), |
| Primer No.29: | |
| CTCCATCGCGAACGGGG | (BTEB juice-1), |
| Primer No.30: | |
| CTCCACCACTCTCCGAGC | (BTEB juice-2). |

All fragments were amplified by Polymerase Chain Reaction (PCR) using a thermal cycler and Pfu polymerase (30-35 cycles of 30″, 95° C./30″, 59° C./72° C. 2′30″), and gel purified before ligating into a desired construct. In addition, most oligonucleotide primers contained a linker sequence with a Pst I restriction enzyme site (CAGATCTGCAG). All fragments were cloned into pBS II cloning vector into the Pst I site. The cHS4 insulator region was previously described (Chung, et al, Cell 74:505-514, 1993). The regulatory DNA fragments can be assembled in a variety of expression vectors, containing basal promoters and reporter genes.

There are numerous sources of information regarding Polymerase Chain Reaction and its applications, and cloning of PCR fragments into vectors. Examples are provided below: Recombinant DNA Laboratory Manual, J, W. Zyskind, S. I. Berstein, Academic Press 1989; Basic Methods in Molecular Biology, L. G. Davis, M. D. Dibner, and J. F. Battey, Elsevier Press 1986; and Directed Mutagenesis, A Practical Approach, M. J. McPherson, Oxford University Press 1991.

### Example 3

### Transient Expression Assays

The constructs can be assayed in either 10T1/2 fibroblast cell lines or Hep3B cells transformed by electroporation and maintained in culture as described in Forsythe, J. A., et al., MCB 16:4604-4613, (1996).

Hep3B cells can be maintained in culture as described below. Plasmid DNA is prepared by using commercial kits (Qiagen) and transfected into cells by electroporation with a Gene Pulser (BioRad) at 260V and 960 μF. Duplicate electroporations are pooled and split onto six tissue culture dishes (60 by 15 mm; Corning) containing 2.2 ml of medium. Cells are allowed to recover for 24 hours in 5% CO2-95% air incubator at 37° C. The cells are given fresh medium, and three plates from each set are transferred to a modular incubator chamber (Billups-Rothenberg, Del Mar, Calif.), which are flushed with 1% O₂-5% CO₂-94% N₂, sealed, and placed at 37° C. The cell are harvested 48 to 72 hours after transfection. Cell pellets are resuspended in 0.25 M Tris HCl (pH 8.0), and extracts are prepared by four freeze-thaw cycles. Protein concentrations are determined by a commercial kit (BioRad), using bovin serum albumin as the standard. β-Galactosidase (β-gal) activity is determined by the hydrolysis of o-nitrophenyl-β-D-galactopyranoside (Promega), using 25 μg of extract at 37° C. for 1 hour, as measured by the A₄₂₀. Lac activity is determined by using 20 μg of cell extract and 100 μl of the lac assay reagent (Promega), which are mixed briefly and placed in a luminometer (Tropix). Light production is measure for 15 s, and results are expressed in relative light units (RLU). Each extract is assayed twice, and the mean RLU is corrected by values obtained from an extract prepared from nontransfected cells. The relative lac activity (mean=standard error of the mean [SEM]) is calculated as lac (RLU)/β-gal (A₄₂₀ per milligram of protein per hour).

### Example 4

### How to Detect an Interaction of a Compound with a Construct as Shown in Example I

The detection of a meaningful interaction of a compound with a target protein or element within a signaling pathway converging on a particular enhancer depends on the nature of the reporter system used. Luciferase reporters would require a luminometer device of some sort. Fluorescent molecules such as GFP and rhodamine would require a device capable of providing light at the excitation wavelength and a photon detector for measuring light at the emission wavelength together with appropriate filters. If the reporter system uses a colorimetric assay, such as the β-galactosidase generation of a blue precipitate from the X-gal substrate, an absorption detector can be used. However, if two separate and insulated reporter genes are used, the reporters must be capable of signaling expression without interfering with detection of the other reporter.

### Example 5

### How to Screen a Library of Compounds

The regulatory DNA assay construct is transfected into an appropriate cell line and an established transfected stable cell line is obtained. The cells can then be grown under selective media and a large quantity thereby attained. The cells are then plated into standard tissue-culture plates, for example 96- or 384-well plates, where multiple individual populations of cells can be maintained separately. The cells are then maintained in these plates until they are ready to be screened in a high-throughput robotic screening (HTS) system, for instance.

Syagen Technology Inc., an analytical instrumentation company has developed a mass spectrometry technology for conducting high-speed molecular analysis. Syagen has adapted this technology to meet critical high-throughput analysis requirements in biopharmaceuticals, drug discovery, and biotechnology. In addition, companies like OBPW make high throughput screening equipment. Also, Aurora's Ultra-High Throughput Screening System (UHTSS) Platform can be used in the present invention. This system is capable of screening up to 100,000 compounds a day in a variety of assays.

The HTS system can be programmed to deliver a wide variety of different compounds at a variety of different concentrations separately into each of the wells of the plate. Then some detection device, such as a fluorescent detection apparatus can be employed to detect the effect of any compound on the cells in any one particular well at various times after application of the compounds. A change in the ratio in two reporters, when two separate reporter systems are included in the assay, will indicate how much the compounds affected one particular pathway versus another, where both pathways converge on two separate enhancers included in the regulatory construct. If the two reporters were GFP and RFP then the individual signals will be detected at different wavelengths by the same detection apparatus and the ratio determined computationally. Analysis of the data should then indicate which well had an interesting signal ratio. Because the allocation of compounds to each numbered well would be recorded, the effective compounds with biologically activity would be known.

Numerous modifications may be made to the foregoing systems without departing from the basic teachings thereof. Although the present invention has been described in substantial detail with reference to one or more specific embodiments, those of skill in the art will recognize that changes may be made to the embodiments specifically disclosed in this application, yet these modifications and improvements are within the scope and spirit of the invention, as set forth in the claims which follow. All publications or patent documents cited in this specification are incorporated herein by reference as if each such publication or document was specifically and individually indicated to be incorporated herein by reference.

Citation of the above publications or documents is not intended as an admission that any of the foregoing is pertinent prior art, nor does it constitute any admission as to the contents or date of these publications or documents.

We claim:

1. An isolated nucleic acid comprising a region that codes for a first regulatory module operably linked to a region that codes for an insulator, said region that codes for an insulator being operably linked to a region that codes for a second regulatory module that is different from said region that codes for the first regulatory DNA module.

2. The nucleic acid of claim 1, wherein said first regulatory module or second regulatory module is an enhancer.

3. The nucleic acid of claim 1, wherein said first regulatory module and second regulatory module are both an enhancer.

4. The nucleic acid of claim 2 or 3, wherein the enhancer is selected from the group consisting of cytoplasmic actin promoter, VEGF hypoxia enhancer, EPO 3′ hypoxia enhancer, LBP-32 enhancer, HMOX1 enhancer, relA hypoxia enhancer, PROC hypoxia enhancer, DELTEX hypoxia enhancer, COL4A1 hypoxia enhancer, GRAP hypoxia enhancer, BTEγ-4 hypoxia enhancer, and the CCRδ5 lymphocyte enhancer.

5. The nucleic acid of claim 1, wherein the insulator is selected from the group consisting of scs, scs′, fab7, fab8, gypsy Su(Hw) array, cHS4 region from the chick globulin locus, and BEAD element.

6. A vector comprising the nucleic acid of claim 1.

7. A vector comprising the nucleic acid of claim 1 wherein a first control module is operably linked to the first regulatory module and a second control module is operably linked to the second regulatory module.

8. The vector of claim 7, wherein a first reporter gene is operably linked to said first control module and a second reporter gene is operably linked to said second control module.

9. The vector of claim 8, wherein said first and second reporter gene are each selected from the group consisting of lacZ and derivatives thereof, luciferase and derivatives thereof, Red Fluorescent Protein (RFP) and derivatives thereof, Green Fluorescent Protein (GFP) and derivatives thereof, blue fluorescent protein and derivatives thereof, cyan fluorescent protein and derivatives thereof, emerald GFP and derivatives thereof, mGFP5er and derivatives thereof, yellow fluorescent protein and derivatives thereof, propidium iodide and derivatives thereof, and alkaline phosphatase and derivatives thereof.

10. The vector of claim 6 in combination with an acellular environment.

11. An isolated, genetically modified cell comprising the vector of claim 9.

12. A method for transfecting a cell with a vector comprising:
a) contacting a cell with the vector of claim 9.

13. A method for constructing a regulatory sequence, which comprises:
a) operably linking a first sequence comprising the coding sequence for a first regulatory module with a second sequence comprising the coding sequence for an insulator;
b) operably linking said second sequence with a third sequence comprising the coding sequence for a second regulatory module, wherein said first and third sequences code for different regulatory modules.

14. A library of isolated nucleic acids each comprising a region that codes for a first regulatory module operably linked to a region that codes for an insulator, said region that codes for an insulator being operably linked to a region that codes for a second regulatory module that is different from said first regulatory module.

15. An isolated nucleic acid comprising a region that encodes a first reporter gene operably linked to a region that codes for a first regulatory module, said first regulatory module being operably linked to a region that codes for an insulator, said region that codes for an insulator being operably linked to a region that codes for a second regulatory module that is different from said first regulatory module, wherein said second regulatory module is operably linked to a region that encodes a second reporter gene that is different from said first reporter gene.

16. The nucleic acid of claim 15, wherein said first regulatory module or second regulatory module is an enhancer.

17. The nucleic acid of claim 15, wherein said first regulatory module and second regulatory module are both an enhancer.

18. The nucleic acid of claim 16 or 17, wherein the first and second enhancers are each selected from the group consisting of cytoplasmic actin promoter, VEGF hypoxia enhancer, EPO 3′ hypoxia enhancer, LBP-32 enhancer, HMOX1 enhancer, relA hypoxia enhancer, PROC hypoxia enhancer, DELTEX hypoxia enhancer, COL4A1 hypoxia enhancer, GRAP hypoxia enhancer, BTEγ-4 hypoxia enhancer, and the CCRδ5 lymphocyte enhancer.

19. The nucleic acid of claim 15, wherein the insulator is selected from the group consisting of scs, scs′, fab7, fab8, gypsy Su(Hw) array, cHS4 region from the chick globulin locus, and BEAD element.

20. The nucleic acid of claim 15, wherein the first and second reporter gene are each selected from the group consisting of lacZ and derivatives thereof, luciferase and derivatives thereof, Red Fluorescent Protein (RFP) and derivatives thereof, Green Fluorescent Protein (GFP) and derivatives thereof, blue fluorescent protein and derivatives thereof, cyan fluorescent protein and derivatives thereof, emerald GFP and derivatives thereof, mGFP5er and derivatives thereof, yellow fluorescent protein and derivatives thereof, propidium iodide and derivatives thereof, and alkaline phosphatase and derivatives thereof.

21. A vector comprising the nucleic acid of claim 15.

22. The vector of claim 21, wherein a first control module is operably linked to said first reporter gene and a second control module is operably linked to said second reporter gene.

23. The vector of claim 21 in combination with an acellular environment.

24. An isolated, genetically modified cell comprising the vector of claim 22.

25. A method for transfecting a cell with a vector comprising:
a) contacting a cell with the vector of claim 22.

26. A method for altering the expression of a reporter gene in a cell comprising:
a) transfecting a cell with the vector of claim 22,
c) culturing the cell under conditions appropriate for expression of the vector; and
d) contacting the cell with one or more compounds.

27. A method for identifying at least one compound that interacts with a test pathway comprising:
a) providing a cell comprising an isolated nucleic acid comprising the coding region for a first reporter gene operably linked to a first control module, said first first control module being operably linked to a first regulatory module, said first regulatory module being operably linked to an insulator sequence, said insulator sequence being operably linked to a second regulatory module different than said first regulatory module, said second regulatory module being operably linked to a second control module, said second control module being operably linked to the coding region for a second reporter gene different than said first reporter gene,
b) contacting the cell with at least one compound;
c) monitoring the differences in the expression levels of the reporter genes, wherein said first reporter gene is operably linked to a control pathway and said second reporter gene is operably linked to a test pathway; whereby
d) a difference in the expression levels of the reporter genes identifies a compound that interacts with the test pathway.

28. An isolated nucleic acid comprising the coding region for a first fluorescent protein operably linked to a first promoter sequence, said first promoter sequence being operably linked to a first enhancer sequence, said first enhancer sequence being operably linked to a cHS4 insulator sequence, said cHS4 insulator sequence being operably linked to a second enhancer sequence, said second enhancer sequence being operably linked to a second promoter sequence, said second promoter sequence being operably linked to the coding region for a second fluorescent protein, wherein the coding region for said first fluorescent protein is different from the coding region for said second fluorescent protein, and said first enhancer sequence is different from said second enhancer sequence.

29. The nucleic acid of claim 28, wherein the first and second enhancers are each selected from the group consisting of cytoplasmic actin promoter, VEGF hypoxia enhancer, EPO 3′ hypoxia enhancer, LBP-32 enhancer, HMOX1 enhancer, relA hypoxia enhancer, PROC hypoxia enhancer, DELTEX hypoxia enhancer, COL4A1 hypoxia enhancer, GRAP hypoxia enhancer, BTEγ-4 hypoxia enhancer, and the CCRδ5 lymphocyte enhancer.

30. The nucleic acid of claim 28, wherein the first and second promoters are each selected from the group consisting of γ-globin promoter and cytoplasmic actin promoter.

31. The nucleic acid of claim 28, wherein the first or second fluorescent protein is selected from the group consisting of green fluorescent protein or derivatives thereof or red fluorescent protein or derivatives thereof.

32. An isolated nucleic acid comprising the coding region for a first fluorescent protein operably linked to a first promoter sequence, said first promoter sequence being operably linked to a first enhancer sequence, said first enhancer sequence being operably linked to a cHS4 insulator sequence, said cHS4 insulator sequence being operably linked to a second enhancer sequence different from said first enhancer sequence, said second enhancer sequence being operably linked to a second promoter sequence, said second promoter sequence being operably linked to the coding region for a second fluorescent protein different from the coding region for said first fluorescent protein, wherein said first or second enhancer sequence is optional.

33. A vector comprising the nucleic acid of any one of claims 28 to 32.

34. A method for altering a protein-protein interaction in a test pathway comprising:
a) providing a cell comprising an isolated nucleic acid comprising the coding region for a first reporter gene operably linked to a first promoter sequence, said first promoter sequence being operably linked to a first enhancer sequence, said first enhancer sequence being operably linked to an insulator sequence, said insulator sequence being operably linked to a second enhancer sequence, said second enhancer sequence being operably linked to a second promoter sequence, said second promoter sequence being operably linked to the coding region for a second reporter gene, wherein the coding region for said first reporter gene is different from the coding region for said second reporter gene, and said first enhancer sequence is different from said second enhancer sequence;
b) contacting the cell with at least one compound;
c) monitoring differences in expression levels of the reporter genes wherein said first reporter gene is operably linked to a control pathway and said second reporter gene is operably linked to a test pathway; whereby
d) a difference in expression levels of the reporter genes identifies a compound that alters a protein-protein interaction in the test pathway.

35. A method for affecting a compound-protein interaction in a test pathway comprising:
a) providing a cell comprising an isolated nucleic acid comprising the coding region for a first reporter gene operably linked to a first promoter sequence, said first promoter sequence being operably linked to a first enhancer sequence, said first enhancer sequence being operably linked to an insulator sequence, said insulator sequence being operably linked to a second enhancer sequence, said second enhancer sequence being operably linked to a second promoter sequence, said second promoter sequence being operably linked to the coding region for a second reporter gene, wherein the coding region for said first reporter gene is different from the coding region for said second reporter gene, and said first enhancer sequence is different from said second enhancer sequence;
b) contacting the cell with at least one compound;
c) monitoring differences in expression levels of the reporter genes wherein said first reporter gene is operably linked to a control pathway and said second reporter gene is operably linked to a test pathway; whereby
d) a difference in expression levels of the reporter genes identifies a compound that alters a compound-protein interaction in a test pathway.

36. An isolated nucleic acid comprising a region that codes for a first regulatory module, said first regulatory module being operably linked to a region that encodes a first reporter gene, said first reporter gene being operably linked to a region that codes for an insulator, said insulator being operably linked to a region that codes for a second regulatory module, wherein said second regulatory module is different from said first regulatory module, and said second regulatory module is operably linked to a region that encodes a second reporter gene that is different from said first reporter gene, and said second reporter gene is linked to a region that codes for an second insulator, said second insulator being different from or the same as said first insulator.

37. The nucleic acid of claim 36 comprising a regulatory module operably linked to a region that encodes for a reporter gene, said region that encodes for a reporter gene being operably linked to a region that codes for an insulator.

38. The nucleic acid of claim 36, wherein said first regulatory module and said second regulatory module are both an enhancer.

39. The nucleic acid of claim 38, wherein said first and second enhancer are each selected from the group consisting of cytoplasmic actin promoter, VEGF hypoxia enhancer, EPO 3′ hypoxia enhancer, LBP-32 enhancer, HMOX1 enhancer, relA hypoxia enhancer, PROC hypoxia enhancer, DELTEX hypoxia enhancer, COL4A1 hypoxia enhancer, GRAP hypoxia enhancer, BTEγ-4 hypoxia enhancer, and the CCRδ5 lymphocyte enhancer, and are not the same.

40. The nucleic acid of claim 36, wherein said first insulator and said second insulator are each selected from the group consisting of scs, scs′, fab7, fab8, gypsy Su(Hw) array, cHS4 region from the chick globulin locus, and BEAD element.

41. The nucleic acid of claim 36, wherein said first reporter gene and said second reporter gene are each selected from the group consisting of lacZ, luciferase, Red Fluorescent Protein (RFP) and derivatives thereof, Green Fluorescent Protein (GFP) and derivatives thereof, and alkaline phosphatase, and are not the same.

42. A vector comprising the nucleic acid of claim 36.

43. An isolated nucleic acid comprising the nucleotide sequence of SEQ ID 1 or any variant thereof.

44. An isolated nucleic acid comprising the nucleotide sequence of SEQ ID 2 or any variant thereof.

45. An isolated nucleic acid comprising the nucleotide sequence of SEQ ID 3 or any variant thereof.

46. An isolated nucleic acid comprising the nucleotide sequence of SEQ ID 4 or any variant thereof.

47. An isolated nucleic acid comprising the nucleotide sequence of SEQ ID 5 or any variant thereof.

48. An isolated nucleic acid comprising the nucleotide sequence of SEQ ID 6 or any variant thereof.

49. An isolated nucleic acid comprising the nucleotide sequence of SEQ ID 7 or any variant thereof.

50. An isolated nucleic acid comprising the nucleotide sequence of SEQ ID 8 or any variant thereof.

51. An isolated nucleic acid comprising the nucleotide sequence of SEQ ID 9 or any variant thereof.
